(19) United States
(12) Patent Application Publication
(10) Pub. No.: US 2012/0220048 A1
(43) Pub. Date: Aug. 30, 2012

(54) MAGNETIC MARKER PARTICLE AND METHOD FOR PRODUCING THE SAME

(75) Inventors: Naoki USUKI, Osaka (JP); Masakazu MITSUNAGA, Osaka (JP); Kenji KONO, Osaka (JP); Hisao KANZAKI, Osaka (JP)

(73) Assignee: HITACHI MAXELL, LTD., Ibaraki-shi (JP)

(21) Appl. No.: 13/151,607

(22) Filed: Jun. 2, 2011

(30) Foreign Application Priority Data
Jun. 3, 2010 (JP) P2010-127728
Jun. 3, 2010 (JP) P2010-127731

(51) Int. Cl. G01N 33/53; H01F 1/01; B82Y 30/00

(52) U.S. Cl. 436/501; 252/62.54; 977/773

(57) ABSTRACT

There is provided a magnetic marker particle. The magnetic marker particle comprises a magnetic particle and a polymer deposited on the surface of the magnetic particle, wherein the deposited polymer comprises a combination of a carboxyl group and a polyethylene glycol chain or a combination of a carboxyl group and a sulfo group.

### TECHNICAL FIELD

The present invention relates to a magnetic marker particle and a method for producing the same. Particularly, the present invention relates to the magnetic marker particle which can be used in the biotechnological field or the life-science field.

### BACKGROUND OF THE INVENTION

In the area of the biotechnology or life-science, a dispersion liquid in which magnetic particles are dispersed has been conventionally used for various kinds of applications such as quantitative analysis, qualitative analysis, separation and purification of cells, proteins, nucleic acids and other biomaterials. Particularly recently, the magnetic particles are used as a marker for detecting target substances (i.e., aimed biological materials). See, Patent Documents 1 and 2 described below, for example.

As a method for synthesizing magnetic particles exhibiting a high dispersion stability, it is known to use an aliphatic carboxylic acid in solvents (see, Patent Document 3 described below). The magnetic particles thus synthesized, however, exhibit a hydrophobic property, thereby showing an extremely poor dispersibility in water. In this regard, when 2-aminoethanol is used, the dispersion stability of these magnetic particles in water can be improved. However, such dispersibility decreases in the neutral range, and thus still providing a problem associated with in the usability (see Non-patent Document 1 described below).

On the other hand, Dynabeads (Registered trademark, manufactured by Invitrogen Corporation) is known as the magnetic beads exhibiting a relatively high dispersion stability in water. However, this magnetic beads are made by including magnetic particles in polymer cores, and thereby having such drawback that a saturation magnetization thereof is not large enough. Moreover, these magnetic beads have a particle size in the range of 1 to 5 μm which is too large to be used as a magnetic marker.

Alternatively, Therma-Max (manufactured by Magnabeat Inc.) is known as a particle having a high dispersion stability and a large amount of magnetization. This Therma-Max particles are coated on their surfaces with a specific coating. However, the usability of such particles is also not satisfactory, since it required to adjust the temperature of the dispersion liquid which contains Therma-Max particles in order to control the dispersion state and the aggregation state of the particles.

In general as for the magnetic particles as described above, the higher dispersibility they have, the less the magnetic collection performance they adversely exhibit. For example, the particles of Patent Document 3 have extremely high dispersibility in a solvent, whereas the magnetic separation thereof can not be performed within a practically acceptable period of time. That is, those particles are not appropriate for the magnetic separation since it takes excessively long time to perform the magnetic separation. On the other hand, when those particles are dispersed in water, the dispersion water shows poor dispersibility, whereas it can afford to perform magnetic separation. In addition, the above-mentioned Dynabeads can afford to perform magnetic separation due to their large particle size, but such particle size thereof is so large to be used as a magnetic marker. Moreover, the above-mentioned Therma-Max can afford to perform magnetic separation by controlling the dispersion state and the aggregation state, but still has a problem in usability as mentioned above.

Patent Document 4 discloses particles exhibiting high dispersion stability and satisfactory magnetic collection performance. However, those properties of Patent Document 4 are merely directed to superparamagnetic particles, and the ferromagnetic particles provide a problem associated with their usability since they are inadequate from a viewpoint of causing magnetic aggregation phenomenon.

With respect to the shapes of the particles to be used in the area of the biotechnology or life-science, the particles in most cases have an irregular shape (that is, a mixed shape made of various particles with various shapes) while they may have a plate-like shape or a rectangular parallelepiped shape. In the case of the irregular shape, the particles can have different surface conditions from each other due to their various shapes, which may cause uneven measurement results when the particles are used as the magnetic marker.

When the magnetic particles are practically used, there may be a problem associated with their behavior that the particles tend to aggregate one another due to the residual magnetization after the application of the magnetic field (such behavior may also be called “magnetic aggregation”). In most cases, the superparamagnetic particles are used in order to solve such a problem. The reason for this is that the superparamagnetic particles do not have a coercive force, thereby exhibiting no residual magnetization, and thus the magnetic aggregation of such particles is not caused under a condition of no magnetic field.

However, the particle diameter of the superparamagnetic particles is not more than 20 nm in a case where the particle is made of iron oxide. This causes such a problem that the magnetic collection can not be performed under a highly dispersed condition of the dispersion liquid of the particles. In this regard, the above-mentioned Therma-Max (manufactured by Magnabeat Inc.) has solved such a problem. Therma-Max, even though being superparamagnetic particles, is somewhat easy to deal with since the particle surfaces thereof are provided with a specific coating, and thereby the dispersion state can vary from high degree to low degree, depending on the temperature. The dispersion liquid containing such particles, however, can not exhibit a satisfactory characteristic in terms of usability, since it required to adjust the temperature of the dispersion liquid in order to control the dispersion state and the aggregation state of the particles.

As such, with respect to the magnetic particles-containing dispersion liquid, there are some restrictions such as the dispersion stability, the magnetic collection performance, the magnetic properties and the particle diameters. Therefore, there is needed a magnetic particle having favorable physical properties, especially having a high dispersion stability and also a high magnetic collection performance. However, as a matter of fact, investigations for such particle and particle dispersion have not been so advanced. In particular, with respect to a pH buffer solution that is often used as a dispersion medium in the area of the biotechnology or life-science, the behavior of the magnetic particles in the pH buffer solution (i.e., dispersion stability of the buffer solution) has not been substantially studied.

### RELATED ART DOCUMENTS

### Patent Documents

[Patent Document 1] Japanese Patent Kohyo Publication No. 2003-524781
[Patent Document 2] Japanese Patent Kokai Publication No. 2005-188950
[Patent Document 3] Japanese Patent Kokai Publication No. 2005-48250
[Patent Document 4] Japanese Patent Kokai Publication No. 60-1564
[Patent Document 5] Japanese Patent Kokai Publication No. 2008-201666
[Patent Document 6] Japanese Patent Kokoku Publication No. 7-6986

### Non-Patent Documents

[Non-patent Document 1] Journal of Magnetism and Magnetic Materials, 320 (2008) L121
[Non-patent Document 2] Water Research and 13 (1979) 21
[Non-patent Document 3] Journal of Colloid and interface Science, 74 (1980) 227
[Non-patent Document 4] Chemistry of Materials, 20 (2008) 198.

### SUMMARY OF THE INVENTION

### Problems to be Solved by the Invention

Under the above circumstances, the present invention has been created. That is, an object of the present invention is to provide a magnetic particle (more specifically, magnetic marker particle) which exhibits an excellent dispersion stability even in the pH buffer solution, and more preferably to provide a magnetic marker particle exhibiting not only a practically satisfactory dispersion stability but also a practically magnetic collection performance in a pH buffer solution.

### Means for Solving the Problem

Through an extensive research, the present inventors have finally focused on the steric structure of the particle and also the compositions of the polymer coating provided on the surface of the magnetic particle, and consequently have found a magnetic marker particle having excellent dispersibility (i.e., degree of dispersion) and dispersion stability even in the buffer solution. Moreover, the inventors also have found a magnetic marker particle exhibiting an excellent magnetic collection performance while having practically no problem in dispersion stability by making consideration for the diameter of aggregated particles. As such, the present invention has been created.

The magnetic marker particle of the present invention is a particle comprising a magnetic particle and a polymer deposited on the surface of the magnetic particle (hereinafter, the polymer may also referred to as “deposited polymer”). In this magnetic marker particle, the deposited polymer comprises a combination of a carboxyl group and a polyethylene glycol chain or a combination of a carboxyl group and a sulfo group. Preferably, the polymer comprises a combination of the carboxyl group, the polyethylene glycol chain and the sulfo group. With respect to a pH buffer dispersion liquid obtained by dispersing the above magnetic marker particles in a pH buffer solution, a value of sedimentation velocity V_{B} (i.e., objective measure I_{S} of dispersion stability as explained in detail below) represented by the following Formula 1 is in the range of about 5.0×10⁻³ to about 6.0, in some cases the range of about 6.0×10⁻³ to about 5.5, or in another cases the range of about 2.3×10⁻² to about 5.0, and thus the magnetic marker particle exhibits a high dispersion stability or a practically satisfactory dispersion stability.

V_{B}=V_{S}/A (Formula 1)

wherein
V_{B} [μm/(s·G)]: Sedimentation velocity of magnetic marker particle in buffer solution;
A[G]: Centrifugal force applied to buffer solution; and
V_{S} [μm/s]: Sedimentation velocity of magnetic marker particle in buffer solution when centrifugal force A is applied thereto.

Specifically, the term “buffer solution” used in the above Formula 1 substantially means a physiological salt solution of phosphoric acid (PBS) with its pH 7.2. Similarly, the “buffer solution” used in the following Formulae 2 and 3 also substantially means a physiological salt solution of phosphoric acid (PBS) with its pH 7.2.

In one preferred embodiment, a sedimentation velocity ratio R is in the range of 1.0 to 18, such ratio being obtained by dividing the value of sedimentation velocity V_{B} of the magnetic marker particles in a case of the particles-containing buffer solution by the value of sedimentation velocity V_{W} of the magnetic marker particle in a case of the particles-containing water (see the following Formula 2):

R=V_{B}/V_{W} (Formula 2)

wherein
R[−]: Ratio of sedimentation velocity value of magnetic marker particle contained in buffer solution to sedimentation velocity value of magnetic marker particle contained in water;
V_{B} [μm/(s·G)]: Sedimentation velocity of magnetic marker particle contained in buffer solution; and
V_{W} [μm/(s·G)]: Sedimentation velocity of magnetic marker particle contained in water.

In general, the dispersion stability tends to decrease in the case of the particles-containing buffer solution, rather than the case of the particles-containing water. Accordingly, the above ratio R makes it possible to evaluate the dispersion stability in a buffer solution while comparing it with the case of water. In this regard, as for the present invention, the value of the ratio R is in the range of 1 to 18. This value is more or less close to 1, and thus the magnetic marker particle of the present invention, when being dispersed in the buffer solution, exhibits substantially the same dispersion stability as that in water. The term “water” used herein means those such as an ion exchanged water, a sterilized water and an ultrapure water. In particular, the term “water” means an ultrapure water.

In another preferred embodiment, a buffer solution containing the magnetic marker particles has a value of sedimentation velocity V′ represented by the following Formula 3 in the range of 1.0×10⁻⁶ to 1.0×10⁻⁴. In some cases, V′ is in the range of 1.0×10⁻⁵ to 8.0×10⁻⁵.

V′=V_{S}/(A×D²) (Formula 3)

wherein
V′[T/m·s·G]=[10¹²/m·s·G]: Sedimentation velocity of magnetic marker particle in buffer solution;
D [nm]: Diameter of magnetic marker particle as primary particle;
A[G]: Centrifugal force applied to buffer solution; and
V_{S} [μm/s]: Sedimentation velocity of magnetic marker particle in buffer solution when centrifugal force A is applied thereto.

It should be noted that the value V_{B} of the above Formula 1 depends on the particle diameter, and that such dependence can be cancelled by dividing the value V_{B} by the square of the particle diameter according to the Stokes' equation. As such, Formula 3 is based on such a concept that the value Vₛ is divided by the square of the primary particle diameter, and thereby the value of the sedimentation velocity V′ is provided while still making consideration for a factor of the degree of the particle aggregation. According to the present invention, the value V′ of the sedimentation velocity regarding the magnetic marker particle, which is represented by Formula 3, is in the range of 1.0×10⁻⁶ to 1.0×10⁻⁴, which indicates that the magnetic marker particle of the present invention has a high dispersion stability or a practically satisfactory dispersion stability.

In the meanwhile, the term “primary particle diameter” means a size of the particle under such a condition that the particles have not yet been dispersed into a buffer solution. Such particle size is provided by measuring each particle size of for example 300 particles on the image of a transmission-type electron microscope photograph or optical microscope photograph, and then calculating the number average thereof.

The magnetic marker particles of the present invention show excellent properties in dispersibility (degree of dispersion) and dispersion stability when being dispersed in a buffer solution. In a particularly preferred embodiment, the magnetic marker particles of the present invention show not only a practically satisfactory dispersion stability, but also a practically satisfactory magnetic-collecting velocity in the buffer solution (i.e., the magnetic marker particle of the present invention exhibits satisfactory properties in terms of dispersion stability and magnetic-collecting characteristics when it is used in the intended use thereof). The expression “practically satisfactory” as used herein means that substantially no problem arises during various operations for various applications (e.g., applications in the test agent for extracorporeal diagnosis, in recovery or test of the biological materials such as DNA and protein in the medicinal and research areas, or in DDS (Drug Delivery System) in the area of the biotechnology or life-science). More specifically, the term “practically satisfactory” substantially means that the magnetic marker particles-containing buffer solution is capable of showing the dispersion stability for at least 10 minutes, or capable of magnetically collecting the magnetic marker particles within 10 minutes therein.

The magnetic marker particles of the present invention are characterized in that the polymer provided on the surfaces thereof comprises “combination of carboxyl group and polyethylene glycol chain” or “combination of carboxyl group and sulfo group”, and thereby the marker particle shows an excellent dispersion stability and dispersibility when dispersed in a buffer solution. In one preferred embodiment, due to the polymer comprising “combination of carboxyl group and polyethylene glycol chain” or “combination of carboxyl group and sulfo group”, the magnetic marker particles of the present invention not only show a practically satisfactory dispersion stability/dispersibility, but also show a practically satisfactory magnetic-collection performance.

As used in this description, the term “magnetic marker particles” substantially means “particles having magnetic properties” which are used in the test agent area for extracorporeal diagnosis, in recovery or test area of the biological materials such as DNA and protein in the medicinal and research, or in DDS (Drug Delivery System) area of the biotechnology or life-science. It is generally desired that the magnetic marker particle is in a single particle form having an average particle diameter of 20 to 500 nm. However, the present invention may also be used in a form of powder (i.e. as group consisting of a plurality of the particles).

As used in this description, the term “buffer solution” or “pH buffer dispersion” means a fluid having a buffering effect which is capable of canceling the pH change upon addition of an acid or a base. More particularly, the term “buffer solution” or “pH buffer dispersion” means a liquid capable of keeping its pH at a nearly constant value thereof, as used in the area of the medical science or bio-science. Especially as for Formulae 1 to 3, the buffer solution means a physiological buffer saline (PBS) of phosphoric acid (pH 7.2).

In this description, the phrase “polymer deposited on the surface of the magnetic particle” substantially covers not only an embodiment wherein the polymer coats the whole surface of the particle body, but also an embodiment wherein the polymer coats on a part of the surface of the particle body”. Preferably, in the magnetic marker particles of the present invention, the deposited polymer is provided on (or adheres to) the surface of the particle body due to a chemical bonding action, not a physical bonding action. As such, the deposited amount of the polymer is relatively low in the magnetic marker particle of the present invention. For example, the amount of the deposited polymer is in the range of 1 to 20% by weight based on the total weight of the magnetic marker particle.

In one preferred embodiment, the deposited polymer comprises a carboxyl group, a polyethylene glycol chain and a sulfo group. Such functional groups and chain can synergistically act with each other and thus effectively contribute to an improved dispersion stability of the particles.

The material for the body of the magnetic marker particle (i.e., material for a core portion of the magnetic marker particle) is not particularly limited as long as the particle is capable of having magnetic properties as a whole. For example, the body of the magnetic marker particle comprises ferrite.

The magnetic marker particle of the present invention can exhibit the practically satisfactory magnetic-collection performance as mentioned above. More specifically, when the magnetic marker particles in a buffer solution are magnetically collected under the magnetic field of about 0.36 T, using the buffer solution containing the magnetic marker particles (the dispersion particle diameter of the magnetic marker particles: about 200 nm to about 700 nm, the concentration of the magnetic marker particles: about 0.1 to 0.3 mg/mL), the time required for the relative light absorbance of the buffer solution to become about 0.1 to about 0.2 is within about 60 seconds (initial value of the light absorbance being 1 before the above magnetic-collection operation).

In one preferred embodiment, the magnetic marker particle of the present invention exhibits an excellent re-dispersion performance (i.e. an excellent dispersibility or dispersion stability even after the magnetic collection). That is, even if the particles have once been aggregated by magnetic collection, the aggregated condition of the particles can be easily dissolved, and thereby making it possible to suitably use the particles again. This performance of the particle may be specifically explained as follows:
When “such a treatment that the magnetic marker particles in the buffer solution are dispersed by ultrasonic irradiation after being magnetically collected” is repeated ten times using the buffer solution containing the magnetic marker particles of the present invention, an increase rate of the dispersion particle diameter of the magnetic marker particles is kept within about 5% from the before-treatment condition.

In one preferred embodiment, the magnetic marker particles of the present invention have a primary particle diameter (i.e., particle diameter in a state before being dispersed into the buffer solution) in the range of 20 nm to 500 nm. Because of having such a particle diameter, the magnetic marker particles of the present invention can show ferromagnetism. In other words, the magnetic marker particles of the present invention are preferably the ferromagnetic particles.

In one preferred embodiment, the magnetic marker particle of the present invention has a biomaterial-binding material and/or a biomaterial-binding functional group immobilized thereon. In other words, the surface of the magnetic marker particle is provided with “substance or functional group that allows the biomaterial (target substance) to bind to the surface of the particle”. Accordingly, when the biomaterial and the magnetic marker particles coexist with each other, the biomaterial can bind to the magnetic marker particles. Thus, the magnetic particles of the present invention can be suitably used as a marker for detecting biomaterials. In this regard, the term “biomaterial (target substance)” means the substances which are conventionally used in the area of the medical science or bio-science. The biomaterials (target substances) may be any suitable substances as long as they can bind to the particle directly or indirectly. Examples of the biomaterial include nucleic acids, proteins (e.g. avidin, biotinylated HRP and the like), sugars, lipids, peptides, cells, eumycetes (fungus), bacteria, yeasts, viruses, glycolipids, glycoproteins, complexes, inorganic substances, vectors, low molecular compounds, high molecular compounds, antibodies, antigens and the like.

The present invention also provides a method for producing the above magnetic marker particle. This method of the present invention is characterized by step of depositing a polymer on the magnetic particle by the use of a polymer raw material wherein the polymer raw material comprises “compound with a polymerizable moiety and a carboxyl group therein”, “compound of a polyethylene glycol chain with at least two polymerizable moieties therein” and “compound with a polymerizable moiety and a sulfo group therein”.

The term “polymerizable moiety” as used in this description substantially means a reactive moiety such as a double bond moiety, a moiety capable of peptide linkage (peptide bonding), and a moiety capable of an amide linkage (amide binding).

In one preferred embodiment of the production method of the present invention, the “compound with a polymerizable moiety and a carboxyl group therein” is an acrylic acid (or acrylic compound), and the “compound with a polymerizable moiety and a sulfo group therein” is a styrenesulfonic acid or a 2-acrylamido-2-methylpropanesulfonic acid.

In the method of the present invention, a commercially available magnetic particle may be used as the magnetic particle serving as a core of the magnetic marker particle. Alternatively, the magnetic particle may be prepared according to the method comprising the steps of:

(i) mixing an iron-containing aqueous solution with an alkaline aqueous solution, thereby precipitating an iron element-containing hydroxide in the resulting mixture solution; and

(ii) subjecting the mixture solution to a heat treatment, thereby forming magnetic particle from the hydroxide.

It is preferred that the method of the present invention further comprises the step of immobilizing a biomaterial-binding material or biomaterial-binding functional group onto the magnetic particle and/or polymer.

The inventors of the present application have additionally studied the particle by focusing not only on “steric structure of the particle and compositions of the polymer coating provided on the surface of the magnetic particle”, but also on “magnetic anisotropy”. This can be explained as follows:

In order to diminish (or decrease) the magnetic aggregation which is problematic from a viewpoint of ensuring a practically satisfactory dispersibility, it is generally necessary to diminish (or decrease) the coercive force. To this end, it is generally necessary to make the particle diameter not more than 20 nm which exhibits superparamagnetic characteristic. Then, it will cause another problem in that the particles do not have a practically satisfactory magnetic collection performance. That is, it is difficult to ensure the practically satisfactory dispersibility, while ensuring the practically satisfactory magnetic collection performance, and thus a trade-off problem is inevitable. Accordingly, the present inventors attempted to address the above problem in a new viewpoint (especially by focusing “magnetic anisotropy”) rather than addressing it in view of an extension of the conventional technology. That is, the present inventors have focused attention on such a matter that the magnetic anisotropy, which could become a factor for the coercive force, should be diminished (or decreased) in order to diminish (or decrease) the coercive force, while keeping the particle diameter capable of the magnetic collection. In this regard, the magnetic anisotropy is classified as two types: “crystalline magnetic anisotropy” caused by geometry of the particles and “structural magnetic anisotropy” caused by the shape of the particles. Since the “crystalline magnetic anisotropy” does not vary depending on the kind of the material, it can be important to decrease the structural magnetic anisotropy attributable to the shape of the particle. The low structural magnetic anisotropy of the particle is considered to be more or less “isotropic”, and in this sense the most isotropic structure is a spherical structure. That is, the present inventors have come up with the conclusion that a particle having low coercive force can be obtained by preparing a particle having a spherical structure. Relating to this matter, some trials intending to prepare a magnetic particle having a spherical shape have long been performed (Patent Document 5, Non-patent Documents 2, 3 and 4), however there remained a problem that the particles have relatively wide particle distributions. Moreover, there is a possibility that a sugar, which was used for the synthesis of the particles, remains on the surface of the particles according to the above Patent Document. Thus, there will arise the problems of unevenness of the surface thereof, and also the non-specific binding phenomenon will occur when the particles are practically used as a magnetic marker.

As such, the marker particle of the present invention created by the inventors through focusing on the “magnetic anisotropy”, has on the one hand, the features of the above marker particles (i.e. magnetic marker particle being characterized by comprising a magnetic particle and a polymer deposited on the surface thereof wherein the polymer comprises a combination of a carboxyl group and a polyethylene glycol chain or a combination of a carboxyl group and a sulfo group), and has on the other hand has a spherical shape wherein a primary particle of the magnetic particle thereof has a ratio of the largest radius to the smallest radius in the range of 1.0 to 1.3 (i.e., so-called “aspect ratio” of the particle is 1.0 to 1.3). In other words, the marker particle of the present invention generally has the approximately spherical shape, and particularly the core magnetic particle thereof has a true spherical shape (true shape).

As described above, the magnetic marker particle of the present invention, which has been created by focusing on “magnetic anisotropy”, has a substantially spherical configuration. That is, such marker particle is a spherical particle. In this regard, the term “spherical configuration” or “spherical” means that the length (or dimension) of a particle is even in every direction thereof and the particle has no anisotropy in size (or in dimension) as a whole. In other words, the magnetic marker particle of the present invention has a true spherical shape wherein a surface shape of the particle has a true spherical shape in terms of geometric configuration. In this context, the term “true sphere” means a sphere wherein a plurality of diameters passing through the center of the sphere have substantially the same length as each other. Specific embodiment regarding this is as follows:

The term “particle having substantially spherical configuration” or “spherical particle” means a particle which has a ratio of the largest radius to the smallest radius in the range of 1.0 to 1.3 (i.e. ratio of the longest dimension to the shortest dimension among the dimensions measured in various directions about the particle being 1.0 to 1.3). Such ratio may be, for example, obtained by measuring the maximum radius value and the minimum radius value about three-hundreds of particles based on a transmission-type electron microscope photograph or an optical microscope photograph of the particles, followed by calculating the ratio thereof.

Especially as for a pH buffer dispersion obtained by dispersing the above spherical magnetic marker particles in a pH buffer solution, the value of sedimentation velocity V_{B} represented by the Formula 1 (i.e. objective measure I_{S} of dispersion stability as explained in detail below) is in the range of about 6.0×10⁻³ to about 4.0, in some cases the range of about 4.0×10⁻³ to about 4.0, or in another cases the range of about 2.3×10⁻² to about 3.5 (for instance, value V_{B} being in the range of about 0.2 to about 2.5 or about 0.5 to about 1.9). Accordingly, the pH buffer dispersion of the spherical magnetic marker particles has a high dispersion stability or a practically satisfactory dispersion stability.

The spherical magnetic marker particles have a sedimentation velocity ratio R of 1.0 to 25, the ratio R being obtained by dividing the value of sedimentation velocity V_{B} of the spherical magnetic marker particles in a case of the particles-containing buffer solution by the value of sedimentation velocity V_{W} of the spherical magnetic marker particle in a case of the particles-containing water. Accordingly, the spherical magnetic marker particles, even in the buffer solution, can have substantially the same dispersion stability as that in water.

As for a pH buffer dispersion obtained by dispersing the spherical magnetic marker particles in a pH buffer solution, the value of the sedimentation velocity V′ represented by the Formula 3 is also in the range of 1.0×10⁻⁶ to 1.0×10⁻⁴ (for instance, V′ in the case of the spherical magnetic marker particle being in the range of 1.0×10⁻⁵ to 8.0×10⁻⁵).

Similarly to the magnetic marker particles described above, when the spherical magnetic marker particles are magnetically collected in a buffer solution under the magnetic field of about 0.36 T, using the buffer solution containing the spherical magnetic marker particles (the dispersion particle diameter of the spherical magnetic marker particles: about 200 nm to about 700 nm, the concentration of the spherical magnetic marker particles: about 0.1 to 0.3 mg/mL), the time required for the relative light absorbance of the buffer solution to become about 0.1 to about 0.2 is within about 60 seconds (initial value of the light absorbance being 1 before the above magnetic-collection operation).

The spherical magnetic marker particle also has an excellent re-dispersion performance (i.e. an excellent dispersibility or dispersion stability even after the magnetic collection). For example, when “such a treatment that the spherical magnetic marker particles in a buffer solution are dispersed after magnetically collected” is repeated, an increase rate of the dispersion particle diameter of the spherical magnetic marker particles is kept at about 2% or less based on the before-treatment condition. It should be noted that “increase rate of the dispersion particle diameter”=“average dispersion particle diameter of the magnetic marker particles after performing the magnetization and re-dispersion treatments”/“average dispersion particle diameter of the magnetic marker particles before performing the magnetization and re-dispersion treatments”×100.

The spherical magnetic marker particles of the present invention have a primary particle diameter (i.e., particle diameter in a state before being dispersed into the buffer solution) in the range of 20 nm to 600 nm. Because of having such a particle diameter, the spherical magnetic marker particles of the present invention can show ferromagnetism. In other words, the spherical magnetic marker particles of the present invention are preferably the ferromagnetic particles.

It is generally desired that the spherical magnetic marker particle is a single particle having an average particle diameter of 20 to 600 nm. However, the present invention may also be used in a form of powder (i.e. as group consisting of a plurality of the spherical particles). In this regard, with regard to the spherical magnetic particles, CV value representing a distribution of their particle diameters is preferably not more than 18%. The term “CV value” as used herein means Coefficient of Variation. More specifically, term “CV value” is a coefficient calculated by statistically processing the whole data of the particle size measurement, and thus is expressed by the following Formula 4:

$$CV value ( \% ) = \frac{\begin{matrix}Standard Deviation of \\ Particle Size Distribution\end{matrix}}{Average Particle Size} \times 100 = \frac{S}{\overline{r}} \times 100 \\ ( \begin{matrix}S \text{:} \begin{matrix}Standard Deviation of \\ Particle Size Distribution\end{matrix} & = & \sqrt{\frac{1}{N} {\sum }_{k = 1}^{N} {( {r}_{k} - \overline{r} )}^{2}} \\ \overline{r} \text{:} Average Particle Size & = & \frac{1}{N} {\sum }_{k = 1}^{N} {r}_{k} \\ {r}_{k} \text{:} Respective Sizes of Particles & & \\ N \text{:} Number of Particles & & \end{matrix} ) \qquad ( Formula 4 )$$

Similarly to the magnetic marker particles described above, each of the spherical magnetic marker particles of the present invention comprises the deposited polymer which is provided on (or adheres to) the surface of the particle body by a chemical bonding action, not by a physical bonding action. As such, the deposited amount of the polymer is relatively low in the spherical magnetic marker particle of the present invention. For example, the amount of the deposited polymer of the spherical magnetic marker particle is in the range of 1 to 20% by weight based on the total weight of the spherical magnetic marker particle.

In one preferred embodiment of the spherical magnetic marker particle, the deposited polymer comprises a carboxyl group, a polyethylene glycol chain and a sulfo group. Such functional groups and chain can synergistically act with each other and thus effectively contribute to an improved dispersion stability of the particles. Moreover, the deposited polymer may comprise a hydroxy group.

The saturation magnetization of the spherical magnetic marker particle is preferably in the range of 2 to 100 A·m²/kg (emu/g). The coercive force of the spherical magnetic marker particle is in the range of about 0.3 kA/m to about 6.5 kA/m (for instance, 0.399 kA/m to 6.38 kA/m). The material for the body of the spherical magnetic marker particle (i.e., material for a core portion of the magnetic marker particle) is not particularly limited as long as the marker particle is capable of having the magnetic properties (especially the above saturation magnetization and/or coercive force) as a whole. For example, the body of the spherical magnetic marker particle comprises ferrite or magnetite.

Similarly to the magnetic marker particle described above, the magnetic particle which constitutes the spherical magnetic marker particle may be prepared according to the method comprising the steps of:

(i) mixing an iron-containing solution with an alkaline solution, thereby precipitating an iron element-containing hydroxide in the resulting mixture solution; and

(ii) subjecting the mixture solution to a heat treatment, thereby forming magnetic particle from the hydroxide. Particularly as for the production method of the spherical magnetic marker particle, it is preferred in the step (ii) that the hydroxide is subjected to a solvothermal reaction in the mixture solution which comprises water and glycerin. It is also preferred that the mixture solution is irradiated with microwave in the heat treatment of the step (ii) (i.e., the microwave is used as a source of heat in the heat treatment of the mixture solution).

Further, the present invention also provides a buffer solution which comprises the magnetic marker particles as described above (i.e., buffer solution with the spherical magnetic marker particles or non-spherical magnetic marker particles therein). This buffer solution of the present invention comprises the above mentioned magnetic marker particles dispersed in a buffer solution medium, and thus exhibits the value of the sedimentation velocity V_{B} represented by the Formula 1 (i.e. objective measure I_{S} of dispersion stability) is in the range of about 5.0×10⁻³ to about 6.0, in some cases the range of about 4.0×10⁻³ to about 5.5, or in another cases the range of about 2.3×10⁻² to about 5.0 (especially as for the buffer solution comprising the spherical magnetic marker particles, the value of V_{B} or I_{S} being in the range of about 6.0×10⁻³ to about 4.0, in some cases the range of about 4.0×10⁻³ to about 4.0, or in another cases the range of about 2.3×10² to about 3.5). Accordingly, the buffer solution of the present invention has a high dispersion stability or a practically satisfactory dispersion stability,

In one preferred embodiment of the buffer solution of the present invention, the value of the sedimentation velocity V′ regarding the magnetic marker particles represented by the Formula 3 is in the range of about 1.0×10⁻⁶ to about 1.0×10⁻⁴, in some cases the range of about 1.2×10⁻⁶ to about 5.0×10⁻⁵, or in another cases the range of about 1.2×10⁻⁶ to about 4.5×10⁻⁵, and thus the buffer solution has a high dispersion stability or a practically satisfactory dispersion stability. Moreover, the buffer solution of the present invention has a sedimentation velocity ratio V_{B}/V_{W} of 1.0 to 18 (i.e. the value R represented by the Formula 2 being 1.0 to 18), obtained by dividing the value of sedimentation velocity V_{B} of the magnetic marker particles in a case of the particles-containing buffer solution by the value of sedimentation velocity V_{W} of the magnetic marker particle in a case of the particles-containing water. Thus, there is little difference between the dispersion stability of the buffer solution of the present invention (i.e. the dispersion stability regarding the magnetic marker particles contained therein) and that in the case of water.

In one preferred embodiment of the buffer solution of the present invention, the dispersion particle diameter of the magnetic marker particles contained therein is in the range of about 200 nm to about 700 nm, and the concentration of the magnetic marker particles is about 0.1 to 0.3 mg/mL, in which case the time period required for the relative light absorbance of the buffer solution becomes about 0.1 to about 0.2 is within about 60 seconds (initial value of the absorbance at point in time before the following magnetic-collection operation being 1) upon magnetically collecting the magnetic marker particles under the magnetic field of about 0.36 T.

In further another preferred embodiment of the present buffer solution, when “such a treatment that the magnetic marker particles are dispersed in the buffer solution by ultrasonic irradiation after being magnetically collected” is repeated ten times, an increase rate of the dispersion particle diameter of the magnetic marker particles is kept within about 5% (particularly as for the buffer solution comprising the spherical magnetic marker particles, the increase rate of the dispersion particle diameter is kept within 2%) compared with that at point in time before the above treatment.

### Effect of the Invention

The magnetic marker particle of the present invention not only has the magnetic properties and particle diameter which are suitable for a marker used in the areas of the medical science and bio-science, but also exhibits an excellent dispersibility (degree of dispersion) and dispersion stability in a pH buffer solution without use of a surfactant. As for the magnetic marker particles each having a spherical shape alone, they have a desired particle diameter distribution, and thus even in this sense they are suitable for using as a marker in the areas of the medical science and bio-science. With regard to the dispersion stability, the value of sedimentation velocity V_{B} (denoted by the Formula 1) regarding the magnetic marker particles of the present invention is in the range of about 5.0×10⁻³ to about 6.0 [μm/(s·G)] (as for that of the magnetic marker particles each having a spherical shape alone, such value of sedimentation velocity V_{B} is in the range of about 6.0×10⁻³ to about 4 [μm/(s·G)]), whereas the value of sedimentation velocity V_{B} regarding the conventional magnetic marker particles in the buffer solution is generally approximately 60 [μm/(s·G)]. In this regard, the value of sedimentation velocity V_{B}, can be regarded as so-called “sedimentation velocity of the dispersed particles in a buffer solution under a static condition” as explained below in detail. Accordingly, the smaller value of the sedimentation velocity V_{B} the magnetic marker particles have, the higher dispersion stability they exhibit. In contrast, the larger value of the sedimentation velocity V_{B} the magnetic marker particles have, the lower dispersion stability they exhibit. In these regards, the larger the dispersion particle diameter becomes, the larger the value of the sedimentation velocity V_{B} becomes. It can be therefore concluded that the dispersion stability of the buffer solution regarding the magnetic marker particles of the present invention is at least ten times higher, more specifically higher by 10 times to 10000 times than that of the conventional magnetic particles having substantially the same primary particle diameter. As for the magnetic marker particles each having a spherical shape alone, it can also be concluded that the dispersion stability of the buffer solution regarding the magnetic marker particles of the present invention is higher by 1.5 times to several thousand times, for example, higher by 2 to 160 times than that of the conventional magnetic particles having substantially the same primary particle diameter.

The sedimentation velocity ratio V_{B}/V_{W}, which is obtained by dividing the value of sedimentation velocity V_{B} of the magnetic marker particles-containing buffer solution by the value of sedimentation velocity V_{W} of the magnetic marker particles-containing water, is in the range of 1.0 to 18 (as for the case of the magnetic marker particles each having a spherical shape alone, the ratio V_{B}/V_{W} is in the range of 1 to 25). This means that the sedimentation velocity of the magnetic marker particles in the buffer solution has substantially little difference from that in water. Furthermore, the value of sedimentation velocity V′ regarding the magnetic marker particles-containing buffer solution, which is denoted by the Formula 3, is in the range of 1.0×10⁻⁶ to 1.0×10⁻⁴. The value V′ is different from the value V_{B} in that the value V′ is obtained by divided the sedimentation velocity by the square of the primary particle diameter. Thus, the value V′ makes it possible to simply evaluate the aggregation conditions of the particles. Even based on this value V′, the dispersion stability of the buffer solution regarding the magnetic marker particles of the present invention is relatively high.

With regard to the dispersibility, “particle diameter of the magnetic marker particles contained in the buffer solution (i.e. the dispersion particle diameter)” measured by dynamic light scattering method (DLS method) is smaller than that of the conventional particles. That is, the aggregation of the particles in the buffer solution is suppressed in accordance with the present invention, and thus the advantage obtained by using the particles with a small particle diameter is not so greatly impaired. Specifically, the dispersion particle diameter “D_{P}” measured in the particles-dispersed buffer solution is slightly higher by approximately 1.1 to 6 times (in some cases by approximately 1.5 to 6 times) than the primary particle diameter D (i.e. “particle diameter of the particles at pint in time before dispersing them into the buffer solution, and visually measured by a microscope”). In view of the fact that the dispersion particle diameter D_{P} measured regarding the conventional particles-containing buffer solution is higher by approximately 6 to 40 times than the primary particle diameter D thereof, it can be concluded that the present magnetic marker particles have a better dispersibility than that of the conventional particles. Moreover, the buffer solution in which the present magnetic marker particles are dispersed shows little variations in the dispersion particle diameters, and can show a superior distribution of the size of particles.

Now, when the dispersion particle diameter D_{P} is too small, the magnetic collectivity tends to decrease while the dispersion stability becomes higher. In other words, the D_{P} is important in terms of the magnetic collectivity, but when D_{P} is too small, a collecting force applied to one aggregating particle becomes small, thereby the particles are hard to collect. While on the other hand, when the dispersion stability is needed, the D_{P} is desired to be as small as possible. That is, the magnetic collectivity generally contradicts the dispersion stability. In this regard, the dispersion particle diameter D_{P} according to the preferred embodiment of the present invention is in the range of 200 to 700 nm, so that both of the magnetic collectivity and the dispersion stability are practically satisfied (when D_{P} falls in the this range, there is no practical problem even if the value D becomes smaller). That is, the present invention is characterized in that both of the magnetic collectivity and the dispersion stability can be satisfied while causing no practical problem and no particular operation in use, by making consideration of the value D_{P}.

In a further preferred embodiment, the magnetic marker particles of the present invention have an excellent re-dispersibility after being magnetically collected, so that they are capable of being re-used in the same application or the other applications.

While not wishing to be bound by any particular theory, the above-mentioned excellent effects and advantages of the magnetic marker particles of the present invention in the buffer solution are due to the characteristic compositions and steric structure thereof (it should be noted that, as for the case of the spherical magnetic marker particles, the advantageous effect is provided by the characteristic compositions and steric structure thereof together with the structural magnetic anisotropy). This can be explained as follows:
In a case where the magnetic particles having only the carboxyl group on the surface thereof are dispersed in a medium, they generally have a high dispersibility due to their electrostatic repulsion caused by the negative charge of the ionized carboxyl group. However, when such particles are dispersed in the pH buffer solution, the negative charge of the carboxyl group will be neutralized by salts contained therein, and thereby the electrostatic repulsion can be diminished. Therefore, the particles with only the carboxyl group on the surface thereof tend to exhibit a decreased dispersibility. In contrast, in a case where the magnetic particles comprise a polymer having not only the carboxyl group but also the polyethylene glycol chain (PEG) therein (i.e., in the case of the present invention), they substantially will be less susceptible to the neutralization attributable to the salts, since PEG has an ether bond portion therein which has a large hydration force, and thus does not have a neutralizable charge. Also in a case where the particles have a sulfo group, they substantially will be less susceptible to the salts contained in the buffer solution, since the sulfo group exhibits a strong acidity and is substantially completely ionized in the solution.
In accordance with the present invention, the PEG chain can have polymerizable groups at both terminals thereof, and thereby being capable of crosslinking between the acrylic chain polymers. This results in a large steric hindrance effect of the particles, which leads to an improved dispersion stability and also an improved “re-dispersibility after the magnetic-collection operation” (see FIG. 3 which will be explained below).

### BRIEF DESCRIPTION OF THE DRAWINGS

FIG. 1 is a flow chart showing processes of the production method of the present invention.

FIG. 2 is photographs each showing the results of “evaluation of dispersion stability” wherein the dispersed states in test tubes at a point in time after allowing them to stand for one month respectively are shown.

FIG. 3 is schematic views wherein “steric hindrances of the particles” resulted from the polyethylene glycol chains are illustrated.

FIG. 4 is a graph showing a dispersion stability from a viewpoint of zeta-potential.

FIG. 5 is schematic views of a measuring embodiment wherein the intensity of the magnetic field is measured in a measuring cell, wherein FIG. 5(a) shows a top view thereof and FIG. 5(b) shows a side view thereof.

FIG. 6 is graphs showing raw data obtained from the implemented measurements of “Evaluation of dispersion stability based on sedimentation velocity”.

FIG. 7 is a graph showing the results of “Evaluation of Magnetic Collectivity”.

FIG. 8 is a graph showing the results of “Evaluation of re-dispersibility”.

FIG. 9 is a graph showing the results of “Evaluation of Magnetic Collectivity” (Specialized in the magnetic marker particles each having a spherical shape).

### BEST MODES FOR CARRYING OUT THE INVENTION

Hereinafter, the magnetic marker particles and the production method therefor according to the present invention will be described in detail.

[symbol]

Magnetic Marker Particles of the Present Invention

[symbol]

Each of the magnetic marker particles of the present invention comprises a magnetic particle or spherical magnetic particle serving as a core (hereinafter, referred also to as a “core particle”) and a polymer deposited on the surface of the core particle wherein the deposited polymer contains “combination of carboxyl group and polyethylene glycol chain” or “combination of carboxyl group and sulfo group”.

The magnetic marker particles of the present invention have magnetic properties as well as a size and shape suitable to be used as a marker in the area of the biotechnology or life-science. Specifically, the magnetic marker particles have a saturation magnetization in the range of 2 A·m²/kg (emu/g) to 100 A·m²/kg (emu/g), preferably in the range of 4 A·m²/kg (emu/g) to 90 A·m²/kg (emu/g). In terms of the magnetic marker particles each having a spherical shape alone, the saturation magnetization thereof is, for example, in the range of 60 A·m²/kg (emu/g) to 80 A·m²/kg (emu/g). When the saturation magnetization of the marker particle falls below the lower limit of the above range, a sensitivity of the particle to the magnetic field tends to decrease, and thereby the magnetic response of the particle decreases. While on the other hand, when the saturation magnetization of the marker particle exceeds the upper limit of the above range, the particles may tend to magnetically aggregate in excess, and thereby the dispersibility of the particles becomes lower. The values of the saturation magnetization in the present specification are those obtained, for example, by measuring the amount of magnetization when a magnetic field of 796.5 kA/m (10 kilo oersted) is applied using a vibration sample magnetometer (manufactured by Toei Kogyo Co., Ltd.). The coercive force of the magnetic marker particles is preferably in the range of 0.079 kA/m to 15.93 kA/m (10 Oe to 200 Oe), more preferably in the range of 1.59 kA/m to 11.94 kA/m (20 Oe to 150 Oe). In terms of the magnetic marker particles each having a spherical shape alone, the coercive force thereof is preferably in the range of 0.399 kA/m to 6.38 kA/m (5 Oe to 80 Oe), more preferably in the range of 0.399 kA/m to 4.79 kA/m (5 Oe to 60 Oe), still more preferably in the range of 0.399 kA/m to 3.19 kA/m (5 Oe to 40 Oe), and as one example thereof, the coercive force of the spherical magnetic marker particle may be in the range of 3.0 kA/m to 4.0 kA/m. The magnetic marker particles may be magnetized to some extent depending on the magnetic field/magnetic flux applied during the magnetic collection. When the coercive force of the particles exceeds the upper limit of the above range, the aggregation force among the particles may increase excessively, and thereby the dispersibility of the particles becomes lower. On the other hand, when the coercive force of the particle falls below the lower limit of the above range, the kinds of the core particles to be used for the marker particles and also the production method of the core particles tend to be limited. The value of “coercive force” as used in this description is a value of the applied magnetic field at which the magnetization amount becomes zero when the magnetic field is returned to zero after applying the magnetic field of 796.5 kA/m (10 kOe), and then the magnetic field is gradually increased in the reverse direction.

As long as the magnetic marker particles of the present invention have the above magnetic properties, the “core particle” used in the present magnetic marker particles may be any suitable particle or any suitable spherical particle. For example, it is preferred that the core particle is not a superparamagnetic particle but a ferromagnetic particle, such as a ferromagnetic oxide particle or a spherical ferromagnetic oxide particle. The term “ferromagnetic” as used herein means such a property that may be substantially permanently magnetized in response to the magnetic field. The term “ferromagnetic oxide particle” as herein means a metal oxide particle which corresponds to a particulate having a magnetic responsibility (i.e., sensitivity to the magnetic field). The phrase “having a magnetic responsibility” means a property having a sensitivity to the magnetic field/magnetic flux, such as being magnetized in response to an external magnetic field/magnetic flux attributable to magnets or the like, or being attracted by the magnets. Examples of the material for the ferromagnetic oxide may include, but not particularly limited to, any known metals such as iron, cobalt and nickel as well as alloys and oxides thereof. In particular, it is preferred that the ferromagnetic oxide particle is a ferromagnetic iron oxide particle since it has an excellent sensitivity to the magnetic field/magnetic flux. As the ferromagnetic iron oxide for such particle, various kinds of known ferromagnetic iron oxides may be used. Particularly, it is preferred that the ferromagnetic iron oxide is at least one ferrite selected from the group consisting of maghemite (γ-Fe₂O₃) magnetite (Fe₃O₄), nickel zinc ferrite (Ni₁₋ₓZnₓFe₂O₄) and manganese zinc ferrite (Mn₁₋ₓZnₓFe₂O₄) since they have an excellent chemical stability. Among them, magnetite (Fe₃O₄) is particularly preferred since it has a large amount of magnetization and an excellent sensitivity to the magnetic field/magnetic flux. Depending on the application or the surface treatment, magnetic metals such as iron and nickel or alloys thereof may also be suitably used.

Many of the magnetic particles which are frequently used in the area of the biotechnology have superparamagnetism. The reason for this is that the superparamagnetic particle has significantly small residual magnetization (remanent magnetization) and coercive force, and thus the superparamagnetic particles, even without being subjected to any particular treatment, rarely affects their re-dispersibility characteristic after the magnetic-collection operation. On the other hand, when a particle having a ferromagnetism, and thereby having coercive force is used, such particle tends to cause a problem associated with magnetic aggregation unless a particular treatment is provided. That is, the ferromagnetism particle having coercive force is hard to use. In general, the primary particle diameter at which the iron oxide (e.g., magnetite) exhibits the superparamagnetism is considered to be less than 20 nm. Thus, the particle having a larger primary diameter than that will exhibit ferromagnetism.

The magnetic marker particles of the present invention preferably has an average particle diameter (primary particle diameter) in the range of about 5 nm to about 1000 nm, more preferably in the range of about 20 nm to about 500 nm, for example in the range of about 20 nm to about 400 nm. In terms of the magnetic marker particles each having a spherical shape alone, the average particle diameter (primary particle diameter) is in the range of about 20 nm to about 6000 nm, preferably in the range of about 20 nm to about 600 nm, more preferably in the range of about 20 nm to about 500 nm, still more preferably in the range of 20 nm to about 400 nm, for example in the range of about 100 nm to about 270 nm. In the case where the particle diameter falls below the lower limit of the above range, the magnetic properties tend to be hardly maintained. On the other hand, in the case where the particle diameter exceeds the upper limit of the above range, a high dispersion stability of the particles-dispersed buffer solution tends to be hardly maintained. As used in this description, the term “particle diameter (particle size)” substantially means a maximum particle length among lengths in all directions of each particle (lengths including a thickness of the deposited polymer). The term “average particle diameter (average particle size)” as used herein substantially means a particle diameter (particle size) calculated as a number average by measuring each particle diameter of 300 particles for example, based on a transmission-type electron micrograph or optical micrograph of the particles.

The density of the magnetic marker particles of the present invention is preferably in the range of 3 to 9 g/cm³, more preferably in the range of 4 to 6 g/cm³. In this regard, the magnetic marker particles of the present invention may have any shape, for example, spherical shape, ellipsoidal shape, rice grain-like shape, acicular shape (or needle-like shape) or plate-like shape. In view of the “structural magnetic anisotropy”, it is however preferred that the magnetic marker particles of the present invention respectively have spherical shapes.

In the magnetic marker particles each having a spherical shape, the shape of the particle is generally spherical one as a whole wherein the ratio of the largest radius to the smallest radius thereof, each of which radius is obtained by measuring the distance from the gravity center to the outer circumference of the particle in various directions, is in the range of 1.0 to 1.3, preferably in the range of 1.0 to 1.25, and more preferably in the range of 1.0 to 1.2. Due to such particle shape with the above ratio of the largest radius to the smallest radius, the structural magnetic anisotropy of the particles (i.e., anisotropy attributable to the particle shape) becomes smaller, and thus the magnetic marker particles have a lower coercive force. In other words, with respect to the spherical particles, not only a practically satisfactory dispersibility but also a practically satisfactory magnetic collectivity is achieved due to the structural magnetic anisotropy thereof together with the characteristic compositions and the steric configurations thereof. In a practical sense, it is difficult to three-dimensionally measure the above ratio (i.e., ratio of the largest radius to the smallest radius of the particle), such ratio is measured from an electron microscope photograph of the particles. As an analysis software for easily obtaining the ratio of the largest radius to the smallest radius of the particle, Image-Pro Plus (manufactured by Nippon Roper Co., Ltd.) is available, in which case a value obtained as “radius ratio” therefrom corresponds to the above ratio (i.e., ratio of the largest radius to the smallest radius of the particle).

As the factors of providing the particle with the coercive force, there are a geometric magnetic anisotropy which depends on the geometric feature (shape), and a crystalline magnetic anisotropy which depends on the material of the particle. In this regard, the spherical magnetic marker particle can make it possible to reduce the geometric magnetic anisotropy thereof. However, the crystalline magnetic anisotropy does not depend on the particle shape, and thus the particle has its intrinsic value thereof according to the material such as maghemite (γ-Fe₂O₃), magnetite (Fe₃O₄), nickel zinc ferrite (Ni₁₋ₓZnₓFe₂O₄) and manganese zinc ferrite (Mn₁₋ₓZnₓFe₂O₄). Accordingly, as long as the particle material generally exhibiting ferromagnetic is used, the coercive force of the particle can not become 0 although it would come close to 0 even if the shape of the particle is spherical. Thus, just because the particle merely has a spherical shape, it does not mean that such particle with a diameter not less than 20 nm exhibits superparamagnetism.

With regard to the magnetic particles each having a spherical shape, CV value (Coefficient of Variation) of the particle diameter thereof is in the range of 0.01% to 19%, preferably in the range of 0.1% to 18%, more preferably in the range of 0.1% to 17%. For example, the CV value regarding the spherical magnetic particles may be in the range of 10% to 17%. The larger the CV value is, the larger the variation in the particle diameters becomes, which may cause the variation of the measurement results when the particle is used as a marker. Thus, the larger CV value is not desired. The term “CV value” as used herein is a coefficient calculated by statistically processing the whole data obtained by the particle size measurement, and is expressed as the following Formula 4. Just as an example, the coefficient of variation of the particle sizes may be obtained for example by measuring the particle sizes of about three-hundreds of particles based on a transmission-type electron microscope photograph or optical microscope photograph of the particles, followed by statistically processing the measured data.

$$CV value ( \% ) = \frac{\begin{matrix}Standard Deviation of \\ Particle Size Distribution\end{matrix}}{Average Particle Size} \times 100 = \frac{S}{\overline{r}} \times 100 \\ ( \begin{matrix}S \text{:} \begin{matrix}Standard Deviation of \\ Particle Size Distribution\end{matrix} & = & \sqrt{\frac{1}{N} {\sum }_{k = 1}^{N} {( {r}_{k} - \overline{r} )}^{2}} \\ \overline{r} \text{:} Average Particle Size & = & \frac{1}{N} {\sum }_{k = 1}^{N} {r}_{k} \\ {r}_{k} \text{:} Respective Sizes of Particles & & \\ N \text{:} Number of Particles & & \end{matrix} ) \qquad ( Formula 4 )$$

According to the present invention, a polymer deposits or adheres to the surface of the core particle. That is, there is a polymer layer on the surface of the magnetic particle serving as the core in the magnetic marker of the present invention. Such polymer layer resides at least in a portion of the core particle surface, preferably resides in the whole surface of the particle such that the polymer layer encloses the core particle. In a particularly preferred embodiment, the polymer layer chemically bonds with the core particle, in which case the amount of the polymer on the magnetic particle is relatively reduced due to such “chemical bond”. Specifically, the amount of the polymer provided in the magnetic marker particles, which may depend on the kinds of the polymer material, can be in the range of 1 to 20% by weight, preferably in the range of 2 to 20% by weight based on the total weight of the magnetic marker particles. In terms of the magnetic marker particles each having a spherical shape alone, the amount of the polymer is in the range of 1 to 20% by weight, preferably in the range of 1 to 10% by weight, more preferably in the range of 1 to 5% by weight based on the total weight of the spherical magnetic marker particles. When the amount of the polymer exceeds the upper limit of the above range, the polymer tends to exist not only merely on the surface of a single core particle, but also exist among a plurality of core particles so that those particles form an aggregate. While on the other hand, when the amount of the polymer falls below the lower limit of the above range, the dispersibility caused by the existence of the polymer will decrease, and thereby a plurality of core particles tend to aggregate one another. The amount of the polymer in the magnetic marker particle can effectively contribute to the “dispersibility and dispersion stability of a buffer solution”, which will be explained infra.

According to the present invention, the polymer deposited on the surface of the core particle (hereinafter, the polymer may also be referred to as “deposited polymer”) contains a combination of a carboxyl group and a polyethylene glycol chain, or a combination of a carboxyl group and a sulfo group (sulfonic acid group) as follow:

### <Carboxyl Group>

—COOH

### <Polyethylene Glycol Chain>

—[CH₂CH₂O]ₙ—

### <Sulfo Group (Sulfonic Acid Group)>

—SO₃H

While not wishing to be bound by any particular theory, the presence of the carboxyl group in the deposited polymer not only provides the particle with hydrophilicity, but also effectively improves the dispersibility and the dispersion stability of the particle in a buffer solution due to an interaction with the polyethylene glycol. The carboxyl group can be introduced into the deposited polymer by using “compound having a polymerizable moiety and a carboxyl group” (e.g., acrylic acid) as a raw material thereof.

Similarly, while not wishing to be bound by any particular theory, when the deposited polymer contains the polyethylene glycol chain, the particle substantially will be less susceptible to the influence of the neutralization attributable to the salt contained in the buffer solution, since the polyethylene glycol chain does not have a neutralizable charge due to the large hydration force of the ether bond portion thereof. In addition, the polyethylene glycol chain is capable of crosslinking between acrylic chin polymers (i.e., carboxyl group-containing polymers), and thereby the particles can have a large steric hindrance effect (see FIG. 3), which can effectively contribute to improved dispersibility and dispersion stability of the particle in the buffer solution. As can be seen particularly from FIG. 3, the polyethylene glycol chain is formed so as to crosslink between the polymers of acrylic backbones, and also the polyethylene glycol chains exist such that they surround the particle as a whole. The polyethylene glycol chain can be introduced into the deposited polymer by using of “compound of polyethylene glycol chain having at least two polymerizable moieties” (e.g., Light-Acrylate available from KYOEISHA CHEMICAL Co., LTD.) as a raw material thereof.

Similarly, while not wishing to be bound by any particular theory, when the deposited polymer contains a sulfo group, it will effectively improves the dispersibility and the dispersion stability of the particles in the buffer solution, since the sulfo group exhibits strong acidity and thus is substantially completely ionized in the solution, making the particles less susceptible to the influence of the salt contained in the buffer solution. The sulfo group can be introduced into the deposited polymer by using of “compound having a polymerizable moiety and a sulfo group” (e.g., styrene sulfonic acid or 2-acrylamido-2-methylpropanesulfonic acid) as a raw material thereof.

As a hydrophilicity-donating group, there are a cationic group and an amphoteric group in addition to an anionic group such as carboxyl group and sulfo group (sulfonic acid group), a nonionic group such as polyethylene glycol. Accordingly, any suitable kinds of groups are usable as long as they provide the same effect as that of the above-mentioned carboxyl group, sulfo group (sulfonic acid group) or polyethylene glycol in the buffer solution.

Examples of the anionic group include compounds having a phosphate group, in addition to the above carboxyl group or sulfo group (sulfonic acid group). The sulfo group (sulfonic acid group) may be one having “—SO₃⁻” at the terminal thereof, and thus the sulfo group may be a sulfonic ester (—OSO₃⁻), a sulphosuccinate (—O₂CCH(CH₂COO⁻)SO₃⁻), a methyltaurine (—CON(CH₃)C₂H₄SO₃⁻) and an isethionic acid (—COOC₂H₄SO₃⁻).

Examples of the cationic group include compounds containing quaternary ammonium salt (e.g., tetraalkylammonium salt) or pyridinium salt, imidazolinium salt.

Examples of the nonionic group include, other than the above-mentioned polyethylene glycol, compounds containing ester (carboxylate —COO—, thioester —(CO—S—), phosphate ester (O═P(O⁻)₃), sulfate ester (—O—SO₂—O—), carbonate ester (—O—C(═O)—O—)), amine oxide (—N(CH₃)₂→O), ether (—O—), hydroxy group (—OH), for example 2-hydroxyethyl acrylate (manufactured by Wako Pure Chemical Industries), 2-hydroxyethyl methacrylate (manufactured by Wako Pure Chemical Industries).

The nonionic compounds do not have a neutralizable charge as with the case of polyethylene glycol, so that the particle substantially will be less susceptible to the influence of the neutralization attributable to the salt contained in the buffer solution.

Examples of the amphoteric group (zwitterionic group) include compounds containing carboxybetaine (R(CH₃)₂N⁺CH₂COO⁻), dimethylamineoxide (R(CH₃)₂NO), sulfobetaine (—N(CH₃)₂C₃H₆SO₃⁻), hydroxy sulfobetaine (—N(CH₃)₂CH₂CH(OH)CH₂SO₃⁻) imidazolinium betaine, beta-aminopropionic acid (—NHC₂H₄COO⁻), for example, carboxymethyl betaine monomer (GLBT) (manufactured by OSAKA ORGANIC CHEMICAL INDUSTRY). Since the charge of the amphoteric compound is in a neutralized state within the monomer molecule thereof, the amphoteric compound substantially will be less susceptible to the influence of the neutralization attributable to the salt contained in the buffer solution. In this regard, however, when the amount of carboxybetaine, dimethylamineoxide would be simply increased, the compound will make it impossible for the particle to bind with avidin as mentioned below, causing the decrease of the performance of the magnetic particle. As such, a suitable amount of the amphoteric compound is needed depending on the usage, and consequently it is necessary to vary the amount of the amphoteric compound according to the intended use.

In the case where the deposited polymer comprises the carboxyl group and the polyethylene glycol chain, the molar ratio of the carboxyl group to the polyethylene glycol chain (i.e., “mole number of the carboxyl group” “mole number of the polyethylene glycol chain”) is preferably in the range of 1:0.001 to 1:0.15, more preferably in the range of 1:0.004 to 1:0.1, for example in the range of 1:0.006 to 1:0.02. In the case where the deposited polymer comprises the carboxyl group and the sulfo group, the molar ratio of the carboxyl group to the sulfo group (i.e., “mole number of the carboxyl group” “mole number of the sulfo group”) is preferably in the range of 1:0.005 to 1:1, more preferably in the range of 1:0.01 to 1:0.1, for example in the range of 1:0.01 to 1:0.04. The term molar ratio in this context is based on an average value from a plurality of magnetic marker particles having a powder form.

The deposited polymer may comprise all of the carboxyl group, the polyethylene-glycol chain and the sulfo group. In this case, the dispersibility and dispersion stability of the particles regarding the buffer solution will be further improved. The molar ratio of the carboxyl group to the polyethylene-glycol chain and to the sulfo group (i.e., “mole number of the carboxyl group”:“mole number of the polyethylene-glycol chain”:“mole number of the sulfo group”) is preferably in the range of 1:0.001:0.005 to 1:0.15:1, more preferably in the range of 1:0.004:0.01 to 1:0.1:0.1, for example in the range of 1:0.006:0.01 to 1:0.02:0.04.

The magnetic marker particles of the present invention preferably comprise “biomaterial-binding material” and/or “biomaterial-binding functional group” immobilized on their surfaces. It is preferred that the biomaterial-binding material is at least one material selected from the group consisting of biotin, avidin, streptavidin and neutravidin. It is preferred that the biomaterial-binding functional group is at least one kind of a functional group selected from the group consisting of carboxyl group, hydroxyl group, epoxy group, tosyl group, succinimide group, maleimide group; sulfide functional groups such as thiol group, thioether group and disulfide group; aldehyde group, azido group, hydrazide group, primary amino group, secondary amino group, tertiary amino group, imide ester group, carbodiimide group, isocyanate group, iodoacetyl group, halogen-substitution of carboxyl group and double bond as well as derivatives thereof. As used in this description, the term “immobilization (immobilized)” substantially means an embodiment wherein “substance to which a target substance can bind” or “functional group to which a target substance can bind” exists in the vicinity of the surface of each core particle and/or deposited polymer. Namely, the term “immobilization (immobilized)” does not necessarily mean only the embodiment wherein “substance to which a target substance can bind” or “functional group to which a target substance can bind” is directly attached to the surface of each core particle and/or deposited polymer. Also, the term “immobilization (immobilized)” substantially means an embodiment wherein “substance or functional group to which a target substance can bind” is immobilized on at least a part of each core particle and/or deposited polymer. Accordingly, “substance or functional group to which a target substance can bind” is not necessarily immobilized over the entire surface of each core particle and/or deposited polymer.

Since the biomaterial-binding materials or functional groups are immobilized on the magnetic marker particles of the present invention, the target substance (i.e., the intended biomaterial) can bind to the particles via such materials or functional groups. As such, the particles of the present invention can be suitably used as the marker particles.

As described above, the magnetic marker particles of the present invention can exhibit an excellent dispersibility/dispersion stability in a pH buffer solution. As used herein, the pH value of the pH buffer solution may be, but not limited to, in the range of about 3 to about 11, preferably in the range of about 5 to about 8. Specific examples of pH buffer solution include acetate buffer solution, phosphate buffer solution, citrate buffer solution, borate buffer solution, tartrate buffer solution, Tris buffer solution, phosphate buffered saline (PBS). These pH buffer solutions are commercially available, but also may be prepared according to any suitable methods. It should be noted that the particles of the present invention provide a particularly advantageous effect in that they exhibit the excellent dispersion stability even in the buffer solution such as the PBS solution which contains a significant amount of salts (KCl/NaCl) therein.

[symbol]

Dispersion Stability and Dispersibility of Magnetic Marker Particles of the Present Invention

[symbol]

“Excellent dispersibility/dispersion stability in a pH buffer solution” as a distinguishing feature of the magnetic marker particles of the present invention will be described in detail.

### (Dispersion Stability Based on a Sedimentation Velocity)

As an index of the dispersion stability, there is a sedimentation velocity of particles in a liquid. Such sedimentation velocity is obtained by a sedimentation condition of the particles after elapse of a given time from point in time when allowing a sample liquid containing the particles to stand. The lower the value of the sedimentation velocity is, the higher the dispersion stability is. The method for measuring the above value generally makes use of the gravity, but it takes time somewhat. In this regard, the sedimentation velocity can be measured under its increased condition by using the centrifugal force, and thereby the measurement time can be shortened. There are LUMiSizer, LUMiFuge (manufactured by Nihon RUFUTO) as the measuring apparatus for carrying out the above method, and thereby a sedimentation velocity V_{S} can be suitably measured. Since these apparatuses are capable of applying a centrifugal force of 2300 G at a maximum, the measurement time can be, in theory, shortened by 2300 times as compared with that of the spontaneous sedimentation. Thus, such apparatuses are very effective for measuring the sedimentation velocity. Moreover, the apparatuses can adjust the centrifugal force in the range of 5G to 2300G. Thus, such a problems that the measurement is difficult due to so high sedimentation velocity or the required measurement time is too long due to so low sedimentation velocity may be solved by selecting a suitable value of centrifugal force for a desired measurement. In this respect, an important matter as to the measurement of the sedimentation velocity of particles is that the sedimentation velocity generally varies depending on the centrifugal force. This can be understood by Stokes' Formula which is a calculation formula for obtaining a rate in a case where small particles settle out in a fluid. That is, it is impossible to directly compare the sedimentation velocities with each other when the applied centrifugal forces are different. Accordingly, the following Formula 1 can evaluate the dispersion stability while eliminating the influence of the centrifugal force:

$${V}_{B} = {V}_{s} / A \\ ( \begin{matrix}{V}_{B} [ \frac{μ m}{( s \cdot G )} ] \text{:} & Sedimentation velocity of magnetic \\ & marker particle in buffer solution \\ A [ G ] \text{:} & Centrifugal force applied to buffer \\ & solution \\ {V}_{s} [ \frac{μ m}{s} ] \text{:} & Sedimentation velocity of magnetic \\ & marker particle in buffer solution \\ & upon applying centrifugal \\ & force A thereto\end{matrix} ) \qquad ( Formula 1 )$$

Formula 1 provides a value which is independent of the centrifugal force, and thereby the dispersion stabilities of the magnetic marker particles in the buffer solution can be directly evaluated.

Here, in the case of the conventional magnetic particles, the value of sedimentation velocity V_{B} regarding a buffer solution is generally about 60 [μm/(s·G)]. While on the other hand, in the case of the magnetic marker particles of the present invention, the value of sedimentation velocity V_{B} is in the range of about 5.0×10⁻³ to about 6.0 [μm/(s·G)] (in terms of the magnetic particles each having a spherical shape alone, the value of sedimentation velocity V_{B} is in the range of about 6.0×10⁻³ to about 4.0 [μm/(s·G)]. As mentioned in the above, the value of sedimentation velocity V_{B} can be substantially identified with “sedimentation velocity of the dispersed particles under a static condition”. The lower the value of sedimentation velocity V_{B} is, the higher the dispersion stability is, and while on the other hand, the higher the value of sedimentation velocity V_{B} is, the lower the dispersion stability is. Accordingly, the value of sedimentation velocity V_{B} (represented by Formula 1) can be identified with the value of the dispersion stability I_{S} of the magnetic marker particles in the buffer solution. In light of this, the dispersion stability of the magnetic marker particles of the present invention in the buffer solution is at least 10 times higher, specifically 10 to 10000 times higher than that of the conventional magnetic particles. In terms of the magnetic marker particles each having a spherical shape alone, the dispersion stability of the magnetic marker particles in the buffer solution is at least 1.5 to several thousand times higher, for example, 2 to 160 times higher, and in a certain case 10 times higher than that of the conventional magnetic particles. In this regard, it is noted that the values of sedimentation velocity V_{B} are those calculated based on the value V_{S} obtained from the measurement using the LUMiSizer, LUMiFuge (manufactured by Nihon RUFUTO).

Comparing the case of the buffer solution with the case of water, the dispersion stability of the particles in the buffer solution is generally lower than that in water. However, in the magnetic marker particles of the present invention, the dispersion stability in the buffer solution does not differ from that in water. More specifically, a ratio R of the sedimentation velocities, which is obtained by dividing the value of sedimentation velocity V_{B} of the magnetic marker particles in the buffer solution by the value of sedimentation velocity V_{W} of the magnetic marker particles in water, is in the range of about 1.0 to about 18 (in terms of the magnetic marker particles each having a spherical shape alone, the ratio R of the sedimentation velocities is in the range of about 1.0 to about 25). See the following Formula 2, wherein V_{W}=[sedimentation velocity (μm/s) of the magnetic marker particles in water, to which centrifugal force A was applied]/[centrifugal force (G) applied to the water].

$$R = {V}_{B} / {V}_{W} \\ ( \begin{matrix}R [ - ] \text{:} & Ratio of sedimentation velocity \\ & value of magnetic marker particle \\ & contained in buffer solution to \\ & sedimentation velocity value of \\ & magnetic marker particle contained \\ & in water \\ {V}_{B} [ \frac{μ m}{( s \cdot G )} ] \text{:} & Sedimentation velocity of magnetic \\ & marker particle contained in \\ & buffer solution \\ {V}_{W} [ \frac{μ m}{( s \cdot G )} ] \text{:} & Sedimentation velocity of magnetic \\ & marker particle contained in \\ & water\end{matrix} ) \qquad ( Formula 2 )$$

As described above, the dispersion stability of the magnetic marker particles of the present invention in the buffer solution does not substantially differ from that in water. In many practical cases it is required to use the buffer solution in which the biomaterials are used, and thus the present particles are desired since they can be used even in the buffer solution in a similar way to that in water.

Furthermore, the dispersion stability of the magnetic marker particles of the present invention is evaluated from a viewpoint of the influence of the particle diameter. The sedimentation velocity V′ which is independent of not only the centrifugal force but also the particle diameter can be denoted by Formula 3 as shown infra (that is, the sedimentation velocity V′ indicates the dispersion stability of the magnetic marker particles, the velocity V′ being independent of the centrifugal force and the particle diameter). When the dispersion particle diameter is used in the formula, the sedimentation velocity would usually become a constant based on the Stokes' Formula. In order to avoid such a matter, Formula 3 makes use of the primary particle diameter. Such value V′ increases as the degree of the aggregation of the particles is higher, and consequently the condition of the aggregation is reflected in Formula 3.

$${V}^{′} = {V}_{s} / ( A \times {D}^{2} ) \\ ( \begin{matrix}{V}^{′} [ \frac{T}{m \cdot s \cdot G} ] = [ \frac{{10}^{12}}{m \cdot s \cdot G} ] \text{:} & Sedimentation velocity of \\ & magnetic marker particle \\ & in buffer solution \\ D [ nm ] \text{:} & Diameter of magnetic \\ & marker particle as \\ & primary particle \\ A [ G ] \text{:} & Centrifugal force applied \\ & to buffer solution \\ {V}_{s} [ \frac{μ m}{s} ] \text{:} & Sedimentation velocity of \\ & magnetic marker particle \\ & in buffer solution upon \\ & applying centrifugal \\ & force A thereto\end{matrix} ) \qquad ( Formula 3 )$$

The magnetic marker particles of the present invention can exhibit the value V′ in the range of about 1.0×10⁻⁶ to about 1.0×10⁻⁴. In light of this value V′, the dispersion stability of the magnetic marker particles of the present invention is high in the buffer solution. Specifically, the dispersion stability of the magnetic marker particles of the present invention is at least 10 times higher than that of the conventional magnetic particles, which is similar to the case of the above Formula 1.

### (Dispersion Stability Based on Zeta-Potential)

The dispersion stability can be evaluated not only from “sedimentation velocity” but also from “zeta-potential”. Such zeta-potential is an important value for generally evaluating the properties of the surface of the particle. In particular, the zeta-potential is an index for evaluating the dispersibility and the aggregability, the mutual interaction, the surface modification of the particles. The magnetic particles become stable as the surface areas thereof are smaller. This gives the magnetic particles a tendency to aggregate each other. On the other hand, the magnetic particles have charge, thereby the electrostatic repulsion acts among the particles. This gives the magnetic particles a tendency to disperse. Since the zeta-potential corresponds to a magnitude of the electrostatic repulsion, it can be used as an index for the stability of the magnetic particles. As the zeta-potential comes close to 0, the tendency of the particles to aggregate each other prevails against the electrostatic repulsion, thereby the aggregation of the particles will be formed. In contrast, the dispersion stability of the magnetic particles may be increased by subjecting the surface of the magnetic particles to the polymer treatment capable of enlarging the absolute value of the zeta-potential (in general, the zeta-potential not less than 20 mV is said to be desired). In this regard, the magnetic marker particles of the present invention exhibits an absolute value of the zeta-potential in the range of 20 to 65 mV, preferably in the range of 30 to 65 mV when they are dispersed in a buffer solution (pH ranging from 3 to 11). Even light of this, the magnetic marker particles of the present invention have an excellent dispersion stability. In this regard, the values of the zeta-potential mentioned in the present specification are those obtained from the measurement using ZetaProbe (manufactured by Nihon Bell). In such apparatus, the value of the zeta-potential can be determined by each variation of the pH value.

### (Dispersibility Based on Dispersion Particle Diameter)

There is “dispersion particle diameter” as an index of the dispersibility of the particles. The dispersion particle diameter is different from the particle diameter obtained from the electron microscope (i.e., different from primary particle diameter). Specifically the dispersion particle diameter is a particle diameter obtained from the Dynamic Light Scattering (DLS) method, and thereby indicating an apparent particle diameter in a buffer solution. Therefore, the dispersion particle diameter indirectly indicates the aggregation condition of the particles in the buffer solution (that is, the degree of the aggregated particles). In other words, as the difference between the primary particle diameter and the dispersion particle diameter is smaller, the degree of the aggregation of the particles is lower and thus the dispersibility thereof is higher (that is, if the primary particle diameter is the same as the dispersion particle diameter, the particles is in a uniform dispersion state wherein respective ones of particles are independently separated from each other in the solution). While on the other hand, as the difference of the dispersion particle diameter from the primary particle diameter is larger, the degree of the aggregation of the particles is higher and the dispersibility thereof is lower. In this respect, the magnetic marker particles of the present invention can have the dispersion particle diameter D_{P} which is approximately 1.1 to 6 times larger than the primary particle diameter D thereof, the D_{P} being measured through dispersing the particles in a buffer solution (pH 3 to 11). In view of the fact that the dispersion particle diameter D_{P} of the conventional magnetic particles in a buffer solution is approximately 6 to 40 times higher than the primary particle diameter D thereof, the magnetic marker particles of the present invention have more excellent dispersibility than that of the prior-art particles.

[symbol]

Magnetic Collectivity of Magnetic Marker Particles of the Present Invention

[symbol]

“Excellent magnetic collectivity in the pH buffer solution”, which is also a distinguishing feature of the present invention, will be described in detail.

As an index of the magnetic collectivity of the magnetic marker particles in the pH buffer solution, “change in light absorbance of the pH buffer solution” may be adopted. That is, the light absorbance measurement through a spectrophotometer can be used for understanding a magnetic collectivity characteristic. This is specifically explained as follows: In a pH buffer solution which contains the magnetic marker particles of the present invention, the magnetic marker particles are dispersed therein so that the pH buffer solution is colored with the color of the magnetic marker particles. When a magnet is brought to approach the dispersion liquid from outside, then the particles with magnetized bodies are forced to gather around the magnet (i.e., the magnetic marker particles are collected near the magnet), thereby the dispersion liquid becomes colorless as a whole. When the light absorbance is measured by means of a spectrophotometer, a high absorbance is shown at the initial dispersion state of the liquid, while the light absorbance gradually becomes lower as the magnetic collection advances. As such, the magnetic collectivity of the particles can be perceived.

The magnetic marker particles of the present invention can exhibit a practically satisfactory magnetic collectivity. This may be quantitatively explained as follows:

When the magnetic marker particles contained in the buffer solution are magnetically collected by a magnetic field of 0.36 T under such a condition that the dispersion particle diameter thereof is preferably in the range of about 200 nm to about 700 nm and the concentration of the magnetic marker particles is for example in the range of about 0.1 to 0.3 mg/mL in the buffer solution containing the magnetic marker particles of the present invention, the time required for the relative light absorbance of the buffer solution to become about 0.1 to about 0.2 is within about 60 seconds (in contrast to the initial value at point in time before the magnetic-collection operation being 1). As an example, in a case where a magnetic field of 0.36 T is applied to a buffer solution in which the dispersion particle diameter of magnetic marker particles is about 350 nm and the concentration of magnetic marker particles is for example about 0.2 mg/mL, the relative light absorbance of the buffer solution (the absorbance of light at about 550 nm) can decrease from its initial value “1” to about “0.15” within about 60 seconds after the initiation of the magnetic collection.

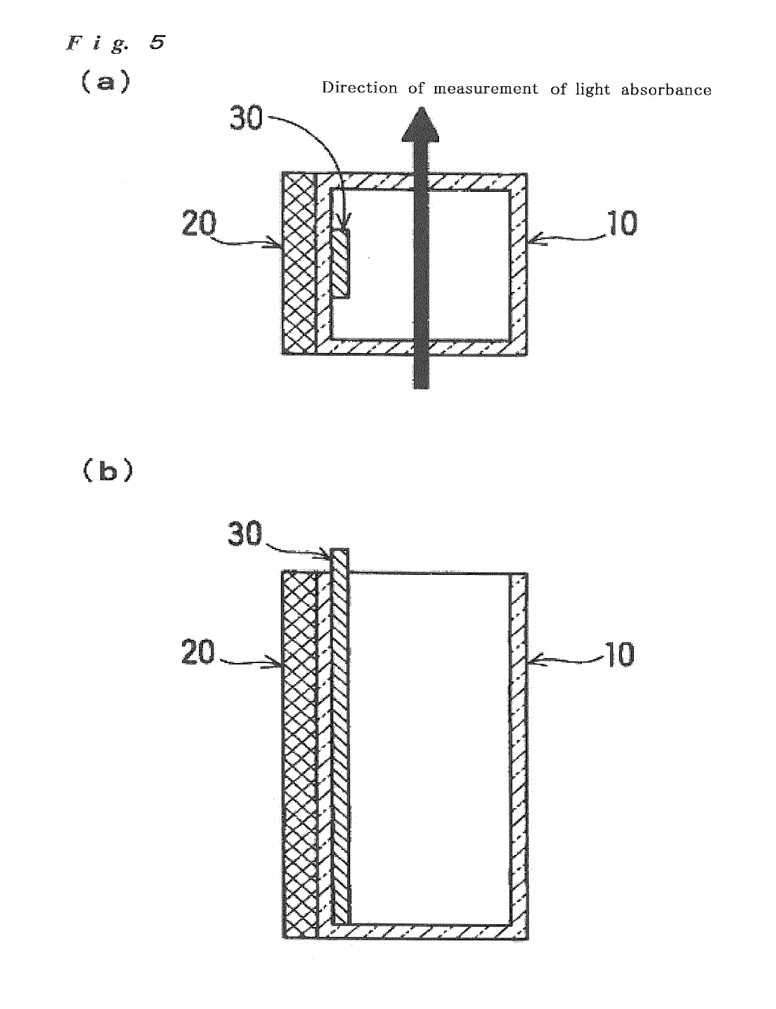

The values of the light absorbance regarding the present invention are those obtained, for example, by using a bio-spectrophotometer U-0080D (manufactured by Hitachi High-Technologies Corporation). As the source of the magnetic field upon the magnetic collection, a magnet can be used in which case any suitable magnets such as a ferrite magnet, a samarium cobalt magnet, a neodymium magnet and an alnico magnet may be used. The value of the magnetic field “0.36 T” is, for example, one measured using Handy Teslameter Elulu DTM6100 (manufactured by Mytech Corporation). A specific embodiment for measuring the intensity of the magnetic field using the above apparatus is shown in FIG. 5. After a magnet is attached to a measurement cell, a sensor assembly is arranged so as to contact with a side-wall of the measurement cell. The tip of the sensor assembly is made contact with the bottom of the side-wall of the measurement cell. As a result, the value of the magnetic field applied to the dispersion can be suitably measured.

When the magnetic collection is performed in a practical use, a strong magnet such as the neodymium magnet, and the samarium cobalt magnet may be used in the application where an accelerated magnetic collecting is desired. In contrast, the ferrite magnet may be used in the application where a delayed magnetic collecting is desired. In another viewpoint, not the material, but the surface magnetic flux density of the magnet may be available as a guide. In such case, the larger the value of the surface magnetic flux density is, the higher the magnetic collecting velocity becomes. While on the other hand, the smaller the value of the surface magnetic flux density is, the lower the magnetic collecting velocity becomes. This value may be determined by the user depending on the intended use. In the practical use, it will be more easily appreciated to measure the intensity of the magnetic field within the measurement cell. In this regard, similar to the above, the higher the intensity of the magnetic field is, the higher the magnetic collecting velocity becomes, whereas, the lower the intensity is, the lower the magnetic collecting velocity becomes. Thus, the value of the intensity of the magnetic field may also be determined by the user depending on the intended use.

### Practically Satisfactory “Dispersion Stability”/“Magnetic Collectivity”

The two properties of “dispersion stability” and “magnetic collectivity” may conflict with each other. In this respect, however, the magnetic marker particles of the present invention preferably have not only a practically satisfactory “dispersion stability”, but also a practically satisfactory “magnetic collectivity”. Specifically, the magnetic marker particles of the present invention have the dispersion particle diameter of about 200 nm to about 700 nm in the buffer solution and the value of sedimentation velocity V_{B} as denoted by the Formula 1 in the range of about 2.3×10⁻² to about 6.0 (in terms of the magnetic marker particles each having a spherical shape alone, the value of sedimentation velocity V_{B} is in the range of about 6.0×10⁻³ to about 4.0, or in the range of about 4.0×10⁻³ to about 4.0, or in the range of about 2.3×10⁻² to about 3.5). In addition, when the magnetic marker particles in a buffer solution are magnetically collected by the magnetic field of about 0.36 T under such a condition that the dispersion particle diameter thereof is in the range of about 200 nm to about 700 nm and the concentration of the magnetic marker particles is in the range of about 0.1 to 0.3 mg/mL in the buffer solution containing the magnetic marker particles of the present invention, the time required for the relative light absorbance of the buffer solution to become about 0.1 to about 0.2 is within about 60 seconds (in contrast to the initial value at point in time before the magnetic-collection operation being 1).

It should be noted that the value of the dispersion particle diameter of the magnetic marker particles in the buffer solution is one obtained for example from measurement using a concentrated particle size analyzer “FPIR-1000” (manufactured by Otsuka Denshi Co., Ltd.). [symbol]
Re-Dispersibility of Magnetic Marker Particles of the Present Invention

[symbol]

The magnetic marker particles of the present invention have an excellent re-dispersibility (i.e. an excellent dispersibility or dispersion stability even after the magnetic collection), too. That is, when the magnetic marker particles are aggregated in the buffer solution by a magnetic collection operation (that is, when subjecting the magnetic marker particles to a magnetization treatment), a suitable dispersion state of the particles can be afterward formed again.

With respect to “re-dispersibility characteristic”, the dispersion particle diameter after the re-dispersing treatment can be regarded as an index therefor. The re-dispersibility can be more excellent as the dispersion particle diameter at point in time after the re-dispersing treatment is closer to that before the re-dispersing treatment. While on the other hand, the re-dispersibility can be unfavorable as the dispersion particle diameter at a point in time after the re-dispersing treatment is larger than that before the re-dispersing treatment. The specific explanation about the re-dispersibility characteristic is as follows: When “such a treatment that the magnetic marker particles are dispersed by ultrasonic irradiation after being magnetically collected” is repeated ten times in a buffer solution, an increase rate of the dispersion particle diameter of the magnetic marker particles (i.e., an increase rate based on the dispersion particle diameter at point in time before performing the magnetization and re-dispersion treatments) is maintained at about 5% or less (that is, the increase rate is in the range of about 0% to about 5%), preferably at about 4% or less (that is, the increase rate is in the range of about 0% to about 4%). In terms of the magnetic marker particles each having a spherical shape alone, the above increase rate is 3% or less (i.e., the increase rate being from about 0% to about 3%), preferably 2% or less (i.e., the increase rate being from about 0% to about 2%), more preferably 1% or less (i.e., the increase rate being from about 0% to about 1%). In this context, the term “magnetic collection” substantially means a treatment for making the magnetic marker particles aggregate in the buffer solution by applying a magnetic field. The term “dispersed by ultrasonic irradiation” substantially means a treatment for re-dispersing the once aggregated magnetic marker particles by ultrasonic irradiation. More specifically, the value of “increase rate of the dispersion particle diameter of the magnetic marker particles” substantially means the value obtained by performing the following magnetic collection and the following ultrasonic irradiation with respect to the following buffer solution:
Buffer solution: medium (phosphate buffered saline (PBS)), particle concentration (10 mg/ml);
Magnetic collection operation: an operation of applying a magnetic field of 0.24 T to the whole buffer solution for 2 minutes (using a stand for separating magnetic beads “Magical Trapper” (manufactured by Toyobo Co., Ltd.), magnetic field measurement apparatus: “Handy Teslameter Elulu DTM6100” (manufactured by Mytech Corporation); and
Ultrasonic irradiation operation (re-dispersion operation): an operation of applying ultrasonic energy to the “area of the aggregated magnetic marker particles” for 2 minutes using an ultrasonic cleaner (VS-150, frequency 50 kHz, output 150 W) (manufactured by As-One Corp.).
It should be noted that the value of the dispersion particle diameter itself is one obtained for example by a measurement using a laser diffraction/scattering particle size distribution analyzer LA-920 (manufactured by Horiba Ltd.).

In light of such a matter that the magnetic marker particles of the present invention may be ferromagnetic particles (that is, the particles generally exhibits the magnetic aggregation characteristic due to “ferromagnetism”), the magnetic marker particles of the present invention have the advantageous features of, on the one hand, having the “ferromagnetism”, and on the other hand, having an excellent “re-dispersibility” (i.e. an excellent dispersibility or dispersion stability even after the magnetic collection operation). The re-dispersibility seems to be resulted from the steric configuration of the particles. While not wishing to be bound by any particular theory, in the magnetic marker particles of the present invention, acrylic polymers are crosslinked one another due to the polymerizable groups provided at each end of the PEG chain, thereby the particles can have a large steric-hindrance effect. Therefore, it is conceived that the “excellent re-dispersibility” is resulted from the “steric-hindrance effect of the particles”. Regarding only to the magnetic marker particles each having a spherical shape, the particles have lower structural magnetic anisotropy (which is attributable to the ratio of the largest radius to the smallest radius of each particle being in the range of 1.0 to 1.3) and thus have lower coercive force, which is also a factor of an improved re-dispersibility.

[symbol]

Production Method of the Present Invention

[symbol]

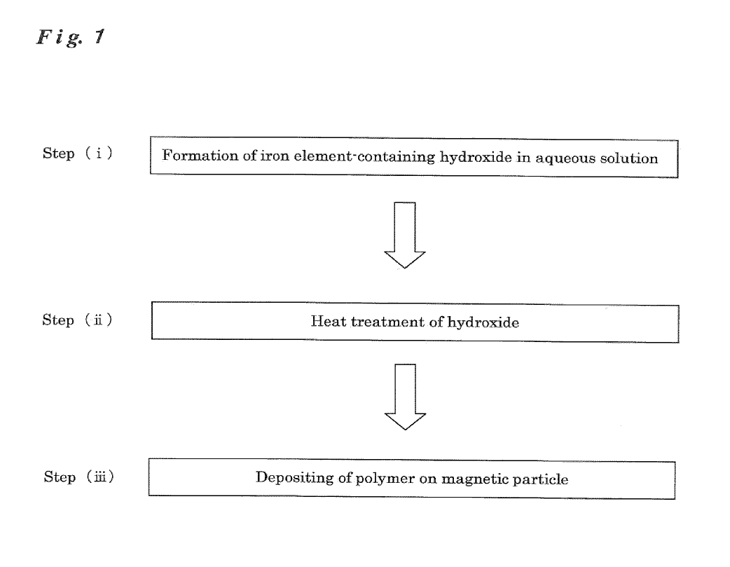

Next, the production method of the present invention will be described. Relating to this, “method of manufacturing magnetic marker particles by preparing magnetic particles (i.e., core particles), followed by producing the intended particles using such magnetic particles” will be described in detail. FIG. 1 is a process flowchart of the production method of the present invention. First, in step (i), an iron ion-containing aqueous solution is mixed with an alkaline aqueous solution, thereby precipitating an iron element-containing hydroxide in the resulting aqueous solution mixture. For example, an alkaline aqueous solution is added to the iron ion-containing aqueous solution. Thereby, an iron ion and an alkaline ion react with each other, and the resulting iron element-containing hydroxide enables it to precipitate in the aqueous solution mixture (such precipitated matter may also be referred to as a “deposited matter” or “coprecipitated matter”).

“Iron ion-containing aqueous solution” to be used in the step (i) is, for example, an acidic aqueous solution obtained by dissolving iron chloride or iron sulfate into water. In this case, the acidic solution generally contains the iron ion. Examples of the iron chloride include ferrous chloride (FeCl₂.4H₂O) and ferric chloride (FeCl₃.6H₂O). Examples of iron sulfate include ferrous sulfate (FeSO₄.7H₂O). Dissolving any of these compounds into water can produce the iron ion. The concentration of the iron ion of the aqueous solution is preferably in the range of 0.03 to 6 mol/l. In order to obtain desired magnetic properties, cobalt ion, platinum ion and/or magnesium ion may be added to the aqueous solution as necessary.

Regarding only to the production method for the magnetic marker particles each having a spherical shape, the iron ion-containing solution to be used is, for example, an aqueous solution obtained by dissolving an iron compound such as iron chloride, iron sulfate and iron acetylacetonato to a solvent capable of dissolving such iron compound. In this case, the iron ion is generally produced in the solution. Examples of the iron chloride include ferrous chloride (FeCl₂.4H₂O) and ferric chloride (FeCl₃.6H₂O), and examples of the iron sulfate include ferrous sulfate (FeSO₄.7H₂O), and examples of the iron acetylacetonato include iron(II) acetylacetonato ((Fe(CH₃COCH═C(O)CH₃)₂). When any of the above compounds is dissolved in a solvent capable of dissolving the compound, the iron ion can generate therein. The compound is dissolved in a solvent capable of readily dissolving the compound, and consequently the solvent is mixed with another solvent which hardly dissolve the compound, and thereby the resulting mixture may be used for the reaction. For example, it is preferred that after the iron sulfate is dissolved in a small quantity of water, the resulting mixture is mixed with a polyhydric alcohol solvent such as glycerin. The glycerin contained in the solution serves to facilitate an isotropic growth of a crystal of the hydroxide (namely, the crystal grows to have a spherical shape). The concentration of the iron ion in the aqueous solution is preferably in the range of 0.03 to 6 mol/l, more preferably in the range of 0.06 to 3 mol/l. As with the above case, in order to obtain desired magnetic properties, cobalt ion, platinum ion and/or magnesium ion can be added to the aqueous solution as necessary.

The alkaline aqueous solution to be used in the step (i) is, for example, an aqueous solution obtained by dissolving an alkaline compound (e.g., NaOH, KOH or NH₃) into water. Therefore, alkali, which is contained in the alkaline aqueous solution, generally exists in the form of an ion. The concentration of the alkali in the alkaline aqueous solution is preferably in the range of 0.03 to 20 mol/l (as for the magnetic marker particles each having a spherical shape alone, the concentration of the alkali in the alkaline aqueous solution is preferably in the range of 0.03 to 20 mol/l, more preferably in the range of 0.06 to mol/l). In this regard, it is preferred that the alkaline aqueous solution contains the alkali ion in an amount corresponding to the ionic valence of iron. It is particularly preferred that an alkali ion exists over the valence of iron ion. If the alkaline ion exists in larger amount than necessary, the number of water washing operation of the resulting ferromagnetic particles will increase, making the washing ineffective.

The temperature condition where an iron ion-containing aqueous solution is mixed with an alkaline aqueous solution is not particularly limited, but may be in the range of about 10° C. to about 90° C. (for example, normal temperature). The mixing operation may be performed under either an aerobic condition or an anaerobic condition. In terms of a simplified operation, the aerobic condition is preferred. There is no particular limitation on the pressure condition during the mixing treatment. For example, the mixing operation may be performed under an atmospheric pressure. With respect to the mixing of “iron ion-containing aqueous solution” and “alkaline aqueous solution”, it is preferable to agitate the iron ion-containing aqueous solution by an agitator such as a magnetic stirrer or three-one motor, while adding dropwise the alkaline aqueous solution by a dropping pump capable of dropping with constant rate.

In the step (ii) of the production method according to the present invention, the aqueous solution mixture obtained from the step (i) is subjected to a heat treatment. The heat treatment may be performed while blowing air into the aqueous solution mixture using an air pump as necessary. It is preferable to control the heating temperature in the range of 70 to 100° C. There is no particular limitation on the pressure condition during the heat treatment. For example, the heat treatment may be performed under an atmospheric pressure. There is also no particular limitation on the heating time period, and for example it may be in the range of about 5 hours to about 12 hours.

Regarding only to the magnetic marker particles each having a spherical shape, it is preferred that the heating temperature of the step (ii) is in the range of 70 to 300° C. There is no particular limitation on the pressure condition during the heat treatment. Thus, the heat treatment may be performed under atmospheric pressure or under a high pressure while heating the pressure container over the boiling point of the solvent therein, which may be referred to as a hydrothermal reaction (or solvothermal reaction). There is also no particular limitation on the heating time period, and for example it may be in the range of 5 hours to 30 hours. There is also no particular limitation on the heating means. For example, any suitable heating devices such as an oil bath, a mantle heater and a dryer may be used, and also another heating device using microwave may be used. With regard to the microwave, there is a limitation on the kind of the solvent to be used since it has to be suitable for the heating of the microwave irradiation. The irradiation of the microwave, however, can provide an advantageous effect in that the solution can be uniformly heated from the inside thereof because the solvent itself is heated. Examples of the heating device using microwave include MicroSYNTH manufactured by Milestone general company.

The heat treatment of step (ii) makes it possible to dissolve the hydroxide and then generate the ferromagnetic iron oxide particles which preferably have spinel structure. Examples of the iron oxide particles having the spinel structure include, but not particularly limited to, magnetite (Fe₃O₄) particles, maghemite (γ-Fe₂O₃) particles, and an intermediate particles of magnetite and maghemite. Depending on the kind of the ions contained in the solution mixture to be subjected to the heat treatment, there can be obtained the above iron oxide particles which further comprise cobalt (Co), platinum (Pt), magnesium (Mg), zinc (Zn) and/or nickel (Ni). The elements such as cobalt, platinum, magnesium and zinc are effective for adjusting the coercive force of the particles. Especially, “addition of cobalt” to the magnetite particles is effective for increasing the coercive force whereas “addition of magnesium” thereto is effective for reducing the coercive force.

It is preferred that the particles formed or synthesized in the step (ii) is subjected to washing, filtration and drying processes. The washing process of the particles make it possible to remove the impurities from the surface thereof. The magnetic particles are washed preferably with water, however may be washed with any suitable solvents capable of being soluble in water, for example alcohol solvents such as ethanol and methanol. The filtration process may be performed together with the washing process, and thereby a wash liquid can be removed from the magnetic particles. The drying process of the particles is not indispensable, and thus, if needed, may be optionally performed. In the case where the drying process is performed, it is preferred that the magnetic particles are dried at a temperature, preferably ranging from 10 to 150° C., more preferably ranging from 40 to 90° C. The magnetic particles may be dried with a dryer, however they may be dried by an air seasoning.

Regarding only to the production method for the magnetic marker particles each having a spherical shape, the steps (i) and (ii) may be performed under either of an aerobic condition or an anaerobic condition. When the reaction is performed under the anaerobic condition, it is necessary to replace the atmosphere in the reactor or the solvent to be used with an anaerobic gas. As the anaerobic gas, various inert gases except for oxygen (e.g., nitrogen or argon) can be used. On the other hand, when the reaction is performed under the aerobic condition, it may be performed under open air.

Through the production steps as described above, the core particles can be obtained. Such core particles preferably may have any suitable shape, for example, spherical shape, ellipsoidal shape, rice grain-like shape, so that the particles will eventually have a desired shape after being subjected to a subsequent process of depositing a polymer. It should be noted that, when the core particles each having a spherical shape are intended to be obtained, the concentration of the alkali is the most contributing factor for forming a spherical shape among the other factors in the present production method. Therefore, the core particles each having a spherical shape can be suitably obtained by optimizing the conditions of the alkali concentration.

Subsequent to the step (ii), the step (iii) is performed. That is, a polymer is deposited on the surface of the magnetic particles by using the raw material thereof. In the case where commercially available magnetic particles are used, the present production method starts from this step (iii). First, the core particles are preferably subjected to a silane coupling agent treatment so as to facilitate the formation of the deposited polymer.

By subjecting the core particles to the silane coupling agent treatment, “polymerizable functional groups (e.g., double bond)” through which the deposited polymer can bind to the surface of the particles are allowed to bind to the core particles. The silane coupling agent which has an acrylic group or methacrylic group on the end thereof may be used. There is no particular limitation on the kind of the solvent for the silane coupling agent treatment as long as the core particles can disperse therein and also the silane coupling agent can dissolve therein. However, the solvent is required to hydrolyze the silane coupling agent, and thus water is required even in a trace amount thereof. Thus, a solvent capable of being miscible with water is preferable. Specifically, it is preferable to use, as the solvent, at least one selected from the group consisting of methanol, ethanol, tetrahydrofuran and water. In order to further promote the hydrolyzation of the silane coupling agent, an acid or an alkali may be added as a catalyst. For example, an acetic acid may be added as an acid catalyst, and an aqueous ammonia may be added as an alkali catalyst. The temperature during the reaction of the silane coupling agent and the core particles can be optionally selected, provided that it is neither below the melting point nor over the boiling point of the solvent to be used. The reaction time period can also be optionally selected, but it is however preferable to select in view of a reaction temperature.

After the silane coupling agent treatment is completed, it is preferable to remove the unreacted silane coupling agent by subjecting the particles to the washing treatment. Although there is no restriction on this washing treatment, a use of the centrifugation technique is simple and thus suitable. After the washing is completed, the core particles may be subjected to a dry treatment. This dry treatment may facilitate to form a chemical bond between the surface of the core particles and the silane coupling agent. Since there is also no particular restriction on this dry treatment, it may be performed at any suitable temperature. For example, a freeze-drying is preferable in order to prevent the aggregation of the particles upon the dry treatment. After the dry treatment is completed, it is required to re-disperse the particles (in this regard, there is also no particular restriction on this re-dispersion of the particles).

The “polymerizable functional groups” on the surface of the core particles, formed through the treatment with the silane coupling agent, is then subjected to a polymer-depositing reaction. Specifically, the core particles, a raw material of the deposited polymer, solvent and an optional polymerization initiator are mixed with each other, and thereby the polymer is allowed to deposit on the surface of the core particles. As the raw material for the deposited polymer, it is preferable to use “compound having a carboxyl group and a polymerizable moiety at its terminal” (e.g. an acrylic acid monomer), “compound having a polyethylene-glycol chain with polymerizable moieties at least at both terminals thereof” (e.g. LIGHT-ACRYLATE manufactured by KYOEISHA CHEMICAL Co., LTD.) and “compound having a sulfo group and a polymerizable moiety at its terminal” (e.g., monomer of 2-acrylamido-2-methylpropanesulfonic acid or styrene sulfonic acid). The solvent for the polymerization may be, but not particularly limited to, at least one selected from the group consisting of water, methanol, ethanol and tetrahydrofuran. Further, the polymerization initiator, which is optionally used as necessary, may be selected according to the kinds of the solvent. For example, in the case where the solvent is water or alcohols, 2,2′-azobis(2-methylpropionamidine) dihydrochloride or a water-soluble azo polymerization initiators such as VA-044 and VA-061 (available from Wako Pure Chemical Industries, Ltd.) may be used.

It is preferable to deposit the polymer on the core particles under such a condition that contains oxygen as little as possible. Thus, the deposition process of the polymer is carried out preferably in a reactor which is charged with the raw materials and also which is filled with nitrogen or argon gas. The temperature for the polymer-depositing process (i.e., reaction temperature) can be optionally set according to a decomposition rate of the reaction initiator. There is no restriction on the time period for performing the polymer-depositing process.

Through such polymer-depositing process, there can be obtained the magnetic marker particles in which the deposited polymer is provided on the surfaces of the core particles. After the polymer-depositing process is completed, the residual polymer which has not deposited to the particles or the unreacted raw monomers are removed from the particles by a washing treatment. Although there is no restriction on this washing treatment, the use of the centrifugation technique is simple and thus suitable.

In the case where the “biomaterial-binding material” or “biomaterial-binding functional group” is immobilized on the surfaces of the magnetic marker particles, such an immobilization treatment may be performed any of before the provision of the deposited polymer, during the provision of the deposited polymer or after the provision of the deposited polymer. For example, in the case where the “biomaterial-binding functional group” is immobilized on the surfaces of the particles after the provision of the deposited polymer, the magnetic marker particles are dispersed in the solvent, and then a compound having the functional group to be immobilized and the reaction catalyst are added to the resulting dispersion liquid under a warmed condition, followed by reacting them for several hours. As a result, the “biomaterial-binding functional group” is immobilized on the surface of the magnetic marker particles. As the solvent to be used in this reaction, any kind of suitable solvent capable of dissolving a compound having the functional group to be immobilized and also capable of providing stable reaction rate even when heated to a temperature over 60° C., may be used. Examples of such solvent include water and ethylene glycol. The catalyst may be used, in which case any kind of suitable catalyst may be used as long as it promotes the above reaction. For example, chloroplatinic acid may be used.

In the case where the immobilization of the “biomaterial-binding functional group” is performed upon the provision of the deposited polymer, a monomer which contains “biomaterial-binding functional group” may be subjected to a polymerization process or a co-polymerization process upon the formation treatment of the deposited polymer. Examples of such monomer include (meth)acrylic acid, glycidyl(meth)acrylate, hydroxyalkyl (meth)acrylate, dimethylaminoalkyl(meth)acrylate, isocyanatoalkyl(meth)acrylate, p-styrenesulfonic acid (p-styrenesulfonate), dimethylolpropanoic acid, N-alkyldiethanolamine, (aminoethylamino)ethanol and lysine. Furthermore, in another case where the “biomaterial-binding material” is immobilized on the surfaces of the magnetic marker particles, a functional group having binding properties to the “biomaterial-binding material” is preliminarily introduced onto the surface of the particle body or the surface of the deposited polymer, and then the “material to which a target substance can bind” can be immobilized to the particle via the preliminarily introduced functional group.

[symbol]

Use of Magnetic Marker Particles

[symbol]

The applications of the magnetic marker particles of the present invention will be additionally described. As described above, the magnetic marker particles of the present invention are those having magnetism which can be used in the applications in the test agent for extracorporeal diagnosis, in recovery or test of the biological materials such as DNA and protein in the medicinal and research areas, or in DDS (Drug Delivery System). As such, the intended biomaterial can be isolated simply by attaching “material capable of specifically binding to such biomaterial” to the surface of the particles, and then mixing the particles with the sample solution, followed by recovering the particles from the solution. This technique may be used in the applications in the test agent for extracorporeal diagnosis, and in recovery or test of the biological materials (e.g., DNA or protein). The magnetic marker particles can be used in the applications in DDS by introducing the particles to which a therapeutic medicine is attached into a body, and thereafter moving the particles to a required portion of the body. In the applications where a sample to be tested in the extracorporeal diagnosis is a body fluid (e.g. blood), or the particles are used for the DDS, the particles of the present invention are extremely useful due to the fact that the blood may be considered as a sort of buffer solution where a significant amount of salts are contained therein.

Although a few embodiments of the present invention have been hereinbefore described, the present invention is not limited to these embodiments and it will be readily appreciated by those skilled in the art that various modifications are possible without departing from the scope of the present invention. For example, although the particles of the present invention have been considered on the assumption that they are used as the marker for detecting an aimed biomaterial (i.e., a target substance), the particles of the present invention can be used for various applications such as quantitative analysis, qualitative analysis, separation or purification of cells, proteins, nucleic acids or other biomaterials, depending on the magnetic properties of the particles, particle sizes or densities thereof (in a case where the particles are used in the separation application of the target substance, the present particles may be referred to also as “particles for magnetic separation”).

It should be noted that the present invention as described above includes the following aspects:

First aspect: A magnetic marker particle comprising a magnetic particle and a polymer deposited on the surface of the magnetic particle,

wherein the polymer comprises a combination of a carboxyl group and a polyethylene glycol chain or a combination of a carboxyl group and a sulfo group (sulpho group); and

wherein a value of sedimentation velocity V_{B} represented by the following Formula 1 with regard to a buffer solution that contains the magnetic marker particle is in the range of 5.0×10⁻³ to 6.0.

$${V}_{B} = {V}_{s} / A \\ ( \begin{matrix}{V}_{B} [ \frac{μ m}{( s \cdot G )} ] \text{:} & Sedimentation velocity of magnetic \\ & marker particle in buffer solution \\ A [ G ] \text{:} & Centrifugal force applied to buffer \\ & solution \\ {V}_{s} [ \frac{μ m}{s} ] \text{:} & Sedimentation velocity of magnetic \\ & marker particle in buffer solution \\ & upon applying centrifugal \\ & force A thereto\end{matrix} ) \qquad ( Formula 1 )$$

Second aspect: The magnetic marker particle according to First aspect, wherein a sedimentation velocity ratio R represented by the following Formula 2 is in the range of 1.0 to 18, the ratio being obtained by dividing the value of sedimentation velocity V_{B} of the magnetic marker particle in a case of buffer solution by the value of sedimentation velocity V_{W} of the magnetic marker particle in a case of water.

$$R = {V}_{B} / {V}_{W} \\ ( \begin{matrix}R [ - ] \text{:} & Ratio of sedimentation velocity \\ & value of magnetic marker particle \\ & contained in buffer solution to \\ & sedimentation velocity value of \\ & magnetic marker particle contained \\ & in water \\ {V}_{B} [ \frac{μ m}{( s \cdot G )} ] \text{:} & Sedimentation velocity of magnetic \\ & marker particle contained in \\ & buffer solution \\ {V}_{W} [ \frac{μ m}{( s \cdot G )} ] \text{:} & Sedimentation velocity of magnetic \\ & marker particle contained in \\ & water\end{matrix} ) \qquad ( Formula 2 )$$

Third embodiment: The magnetic marker particle according to First or Second aspect, wherein a value of sedimentation velocity V′ represented by the following Formula 3 with regard to a buffer solution that contains the magnetic marker particle is in the range of 1.0×10⁻⁶ to 1.0×10⁻⁴.

$${V}^{′} = {V}_{s} / ( A \times {D}^{2} ) \\ ( \begin{matrix}{V}^{′} [ \frac{T}{m \cdot s \cdot G} ] = [ \frac{{10}^{12}}{m \cdot s \cdot G} ] \text{:} & Sedimentation velocity of \\ & magnetic marker particle \\ & in buffer solution \\ D [ nm ] \text{:} & Diameter of magnetic \\ & marker particle as \\ & primary particle \\ A [ G ] \text{:} & Centrifugal force applied \\ & to buffer solution \\ {V}_{s} [ \frac{μ m}{s} ] \text{:} & Sedimentation velocity of \\ & magnetic marker particle \\ & in buffer solution upon \\ & applying centrifugal \\ & force A thereto\end{matrix} ) \qquad ( Formula 3 )$$

Fourth aspect: The magnetic marker particle according to any one of First to Third aspects, wherein the amount of the polymer is in the range of 1 to 20% by weight based on the weight of the magnetic marker particle.
Fifth aspect: The magnetic marker particle according to any one of First to Fourth aspects, wherein the magnetic marker particle is a ferromagnetic particle.
Sixth aspect: The magnetic marker particle according to any one of First to Fifth aspects, wherein the polymer comprises the carboxyl group, the polyethylene glycol chain and the sulfo group.
Seventh aspect: The magnetic marker particle according to any one of First to Sixth aspects, wherein the magnetic particle comprises a ferrite.
Eighth aspect: The magnetic marker particle according to any one of First to Seventh aspects, wherein a biomaterial-binding material or biomaterial-binding functional group is immobilized on the magnetic particle and/or the polymer.
Ninth aspect: The magnetic marker particle according to any one of First to Eighth aspects, wherein the magnetic marker particle has a primary particle diameter of 20 nm to 500 nm.
Tenth aspect: The magnetic marker particle according to any one of First to Eighth aspects, wherein, with respect to a buffer solution containing the magnetic marker particles (dispersion particle diameter of the magnetic marker particles: 200 nm to 700 nm, concentration of magnetic marker particles: 0.1 to 0.3 mg/mL), a time required for relative light absorbance of the buffer solution to become 0.1 to 0.2 (from an initial value being “1” before the following magnetic collection operation) when the magnetic marker particles are magnetically collected in the buffer solution under the magnetic field of 0.36 T is within 60 seconds.
Eleventh aspect: The magnetic marker particle according to any one of First to Ninth aspects, wherein an increase rate of a dispersion particle diameter of the magnetic marker particles contained in a buffer solution is within 5% with respect to the dispersion particle diameter of the magnetic particles contained in the before-treatment buffer solution, provided that the treatment where the magnetic marker particles are dispersed in the buffer solution by an ultrasonic irradiation after being magnetically collected is repeated ten times.
Twelfth aspect: A method for producing the magnetic marker particle according to Sixth aspect, comprising the step of depositing a polymer on the magnetic particle by the use of a polymer raw material,

wherein the polymer raw material comprises “compound with a polymerizable moiety and a carboxyl group therein”, “compound of a polyethylene glycol chain with at least two polymerizable moieties therein” and “compound with a polymerizable moiety and a sulfo group therein”.

Thirteenth aspect: The method according to Twelfth aspect, wherein the “compound with a polymerizable moiety and a carboxyl group therein” is an acrylic acid and the “compound with a polymerizable moiety and a sulfo group therein” is a styrenesulfonic acid or a 2-acrylamido-2-methylpropanesulfonic acid.
Fourteenth aspect: The method according to Twelfth or Thirteenth aspect, wherein the magnetic particle is prepared by a treatment comprising the steps of_{:}

(i) mixing an iron-containing solution with an alkaline solution, thereby precipitating (depositing) an iron element-containing hydroxide in the resulting mixture solution; and

(ii) subjecting the mixture solution to a heat treatment, thereby forming magnetic particle from the hydroxide.

Fifteenth aspect: A magnetic marker particle comprising a magnetic particle and a polymer deposited on the surface of the magnetic particle,

wherein the magnetic marker particle has a spherical shape wherein a ratio of the largest radius to the smallest radius regarding a primary particle thereof is in the range of 1.0 to 1.3.

Sixteenth aspect: The magnetic marker particle according to Fifteenth aspect, wherein the polymer not only comprises a carboxyl group, but also comprises a polyethylene glycol chain or a sulfo group (sulpho group).
Seventeenth aspect: The magnetic marker particle according to Fifteenth or Sixteenth aspect, wherein, with regard to the spherical magnetic particles, Coefficient of Variation (CV value) which represents a distribution of their particle diameters is not more than 18%.
Eighteenth aspect: The magnetic marker particle according to any one of Fifteenth to Seventeenth aspects, wherein a value of sedimentation velocity V_{B} represented by the following Formula 1 with regard to a buffer solution that contains the spherical magnetic marker particle is in the range of 6.0×10⁻³ to 4.0.

$${V}_{B} = {V}_{s} / A \\ ( \begin{matrix}{V}_{B} [ \frac{μ m}{( s \cdot G )} ] \text{:} & Sedimentation velocity of magnetic \\ & marker particle in buffer solution \\ A [ G ] \text{:} & Centrifugal force applied to buffer \\ & solution \\ {V}_{s} [ \frac{μ m}{s} ] \text{:} & Sedimentation velocity of magnetic \\ & marker particle in buffer solution \\ & upon applying centrifugal \\ & force A thereto\end{matrix} ) \qquad ( Formula 1 )$$

Nineteenth aspect: The magnetic marker particle according to any one of Fifteenth to Seventeenth aspects, wherein a value of sedimentation velocity V′ represented by the following Formula 3 with regard to a buffer solution that contains the magnetic marker particle is in the range of 1.0×10⁻⁶ to 1.0×10⁻⁴.

$${V}^{′} = {V}_{s} / ( A \times {D}^{2} ) \\ ( \begin{matrix}{V}^{′} [ \frac{T}{m \cdot s \cdot G} ] = [ \frac{{10}^{12}}{m \cdot s \cdot G} ] \text{:} & Sedimentation velocity of \\ & magnetic marker particle \\ & in buffer solution \\ D [ nm ] \text{:} & Diameter of magnetic \\ & marker particle as \\ & primary particle \\ A [ G ] \text{:} & Centrifugal force applied \\ & to buffer solution \\ {V}_{s} [ \frac{μ m}{s} ] \text{:} & Sedimentation velocity of \\ & magnetic marker particle \\ & in buffer solution upon \\ & applying centrifugal \\ & force A thereto\end{matrix} ) \qquad ( Formula 3 )$$

Twentieth aspect: The magnetic marker particle according to any one of Fifteenth to Nineteenth aspects, wherein an increase rate of a dispersion particle diameter of the spherical magnetic marker particles contained in a buffer solution is within 2% with respect to the dispersion particle diameter of the spherical magnetic particles contained in the before-treatment buffer solution, provided that the treatment where the spherical magnetic marker particles are dispersed in the buffer solution by an ultrasonic irradiation after being magnetically collected is repeated.
Twenty-first aspect: The magnetic marker particle according to any one of Fifteenth to Twentieth aspects, wherein a saturation magnetization of the spherical magnetic marker particle is in the range of 2 to 100 A·m²/kg (emu/g).
Twenty-second aspect: The magnetic marker particle according to any one of Fifteenth to Twenty-first aspects, wherein a coercive force of the spherical magnetic marker particle is in the range of 0.3 kA/m to 6.5 kA/m.
Twenty-third aspect: The magnetic marker particle according to any one of Fifteenth to Twenty-second aspects, wherein the amount of the deposited polymer is in the range of 1 to 20% by weight based on the weight of the magnetic marker particle.
Twenty-fourth aspect: The magnetic marker particle according to any one of Fifteenth to Twenty-third aspects, wherein the spherical magnetic marker particle has a primary particle diameter of 20 nm to 600 nm.
Twenty-fifth aspect: The magnetic marker particle according to any one of Fifteenth to Twenty-fourth aspects, wherein the magnetic particle comprises ferrite or magnetite.
Twenty-sixth aspect: The magnetic marker particle according to any one of Fifteenth to Twenty-fifth aspects, wherein a biomaterial-binding material or biomaterial-binding functional group is immobilized on the magnetic particle and/or the polymer.
Twenty-seventh aspect: The magnetic marker particle according to any one of Fifteenth to Twenty-sixth aspects, wherein the polymer comprises the carboxyl group, the polyethylene glycol chain and the sulfo group.
Twenty-eighth aspect: A method for producing the magnetic marker particle according to Twenty-seventh aspect, comprising the step of depositing a polymer on the magnetic particle by the use of a polymer raw material,

wherein the polymer raw material comprises “compound with a polymerizable moiety and a carboxyl group therein”, “compound of a polyethylene glycol chain with at least two polymerizable moieties therein” and “compound with a polymerizable moiety and a sulfo group therein”.

Twenty-ninth aspect: The method according to Twenty-eighth aspect, wherein the “compound with a polymerizable moiety and a carboxyl group therein” is an acrylic acid and the “compound with a polymerizable moiety and a sulfo group therein” is a styrenesulfonic acid or a 2-acrylamido-2-methylpropanesulfonic acid.
Thirtieth aspect: The method according to Twenty-eighth or Twenty-ninth aspect, wherein the magnetic particle is prepared by a treatment comprising the steps of:

(i) mixing an iron-containing solution with an alkaline solution, thereby precipitating (depositing) an iron element-containing hydroxide in the resulting mixture solution; and

(ii) subjecting the mixture solution to a heat treatment, thereby forming magnetic particle from the hydroxide.

Thirty-first aspect: The method according to Thirtieth aspect, wherein, in the step (ii), the hydroxide is subjected to a solvothermal reaction in the mixture solution which comprises water and glycerin.
Thirty-second aspect: The method according to Thirtieth or Thirty-first aspect, wherein the mixture solution is irradiated with microwave in the heat treatment of the step (ii).
Thirty-third aspect: The method according to any one of Twenty-eighth to Thirty-second aspects, further comprising the step of immobilizing a biomaterial-binding material or biomaterial-binding functional group on the magnetic particle and/or the polymer.

### EXAMPLES

Hereinafter, various kinds of examples regarding the present invention will be explained. Especially, “case specialized in the magnetic marker particles each having a spherical shape” and “case not specialized in the magnetic marker particles each having a spherical shape” are separately explained. First, the case (A) “not specialized in the magnetic marker particles each having a spherical shape” is explained, and then the case (B) “specialized in the magnetic marker particles each having a spherical shape” will be explained.

Buffer solution used in each of cases (A) and (B) is phosphate buffered saline (PBS). This PBS was prepared by dissolving 0.210 g of disodium hydrogenphosphate heptahydrate, 0.031 g of potassium dihydrogen phosphate, and 0.877 g of sodium chloride in 100 ml of water. The pH was 7.2.

### “Case (A): Not Specialized in the Magnetic Marker Particles Each Having a Spherical Shape”

[symbol] Preparation of Particles

[symbol]

In Examples 1 to 20 and Comparative Examples 1 to 5, particles were prepared in the following manner:

### Example 1

### Synthesis of Magnetite Particles

Magnetite particles serving as the core particles were synthesized according to the procedures as follows:

First, 100 g of ferrous sulfate (FeSO₄.7H₂O) was dissolved in 1000 cc of pure water to form an aqueous solution of ferrous sulfate. In 500 cc of pure water, 28.8 g of sodium hydroxide was dissolved so as to be equimolar with the above ferrous sulfate, thereby an aqueous solution of sodium hydroxide was prepared. Then, the aqueous solution of sodium hydroxide was added dropwise to the aqueous solution of ferrous sulfate while stirring the ferrous sulfate solution, and thereby allowing a ferrous hydroxide to precipitate therein. Subsequent to the completion of the dropwise addition of the sodium hydroxide solution, the resulting suspension containing the precipitate of ferrous hydroxide was heated up to 85° C. while stirring the resultant suspension. After the temperature of the suspension reached 85° C., it was subjected to an oxidation treatment for 8 hours while blowing air therein at a rate of 200 L/hr using an air pump, and thereby magnetite particles was formed therein. The magnetite particles each had almost spherical shape and had a primary particle diameter of 24 nm (the primary particle diameter of the magnetite particles was obtained as a number average of 300 particles after measuring each size thereof from a micrograph of transmission-type electron microscope).

### <Silane Coupling Agent Treatment>

2 g of magnetite particles were dispersed in 600 ml of methanol. To the resulting suspension, 20 ml of 3-methacryloxypropyl trimethoxysilane (LS-3360, manufactured by Shin-Etsu Chemical Co., Ltd.) was added and stirred at 40° C. for 4 hours. Subsequently, the suspension was subjected to a centrifugal treatment and washed, and then the solvent medium was replaced with water. As a result, there was obtained the magnetic particles with the silane coupling agent deposited on the surfaces thereof.

### <Depositing Treatment of Polymer>

200 mg of the magnetic particles having the deposited silane coupling agent thereon were dispersed in 60 ml of water. The resulting dispersion was stirred while blowing nitrogen gas thereinto so as to prepare a nitrogen atmosphere. Thereafter, 0.68 g of acrylic acid (manufactured by Wako Pure Chemical Industries, Ltd.), 74 of Light-Acrylate 9EG-A (hereinafter referred to as “PEG”) (manufactured by KYOEISHA CHEMICAL Co., LTD.), 70 mg of 2-acrylamido-2-methylpropanesulfonic acid (hereinafter referred to as “AMPS”) (manufactured by Wako Pure Chemical Industries, Ltd.) were added to the dispersion. While stirring the dispersion for a while, 1.2 mg of 2,2′-azobis(2-methylpropionamidine)dihydrochloride (manufactured by Wako Pure Chemical Industries, Ltd.) was added thereto and reacted under the nitrogen atmosphere at 70° C. for 5 hours. Then, the particles were washed by using the centrifugation technique. As a result, the magnetic marker particles with the deposited polymer thereon were obtained. The particle size of these particles was calculated based on the electron microscope micrograph so as to obtain the “primary particle diameter”. The primary particle diameter of the magnetic marker particle was 24 nm.

### <Measurement of Dispersion Particle Diameter and Amount of Deposited Polymer>

Together with measuring the amount of the deposited polymer, the dispersion particle diameter was measured according to DLS method through dispersing the magnetic marker particles in the buffer solution. The measurement of the amount of deposited polymer was performed according to the thermogravimetric method after the magnetic marker particles were dried. Specifically, the amount of deposited polymer was measured from the loss in weight of the particles upon combustion of the organic materials (polymer) using a thermogravimetric analyzer TG-DTA 2000S (manufactured by Macscience). As a result, the amount of deposited polymer was 15.4% by weight and the dispersion particle diameter was 154.3 nm.

### Examples 2 to 10

The procedure as with Example 1 was performed except that the depositing treatment of polymer was carried out under the condition as shown in Table 1 infra.

### Examples 11-14

Instead of Light-Acrylate 9EG-A (manufactured by KYOEISHA CHEMICAL Co., LTD.), Light-Acrylate 3EG-A (manufactured by KYOEISHA CHEMICAL Co., LTD.) having different length of polyethylene glycol chain was used. Since this Light-Acrylate 3EG-A has low solubility in water, the procedure was carried out in a mixture solvent of water and methanol. Except these, the procedure was carried out as with that of Example 1. The conditions used in procedures of Examples 11-14 are shown in Table 1 infra.

### Example 15

Instead of Light-Acrylate 9EG-A (manufactured by KYOEISHA CHEMICAL Co., LTD.), Light-Acrylate 14EG-A (manufactured by KYOEISHA CHEMICAL Co., LTD.) having different length of polyethylene glycol chain was used. Except this, the procedure was carried out as with that of Example 1. The conditions used in the procedure of Example 15 are shown in Table 1 infra.

### Example 16

The procedure as with Example 1 was performed except that Light-Acrylate 9EG-A (manufactured by KYOEISHA CHEMICAL Co., LTD.), was not used. The conditions used in the procedure of Example 16 are shown in Table 1 infra.

### Examples 17-20

In each of Examples 17-20, the procedure as with Example 1 was performed except that Magnetite TM-023 (manufactured by Toda Kogyo K.K.) (primary particle diameter: 230 nm) was used as the core particle and the amount of the monomer was changed as shown in Table 1.

### Comparative Example 1

The procedure as with Example 1 was performed except that the depositing treatment of polymer was carried out using only the 1.6 g of acrylic acid, not using Light-Acrylate 9EG-A and 2-acrylamido-2-methylpropanesulfonic acid.

### Comparative Examples 2 and 3

In each of Comparative examples 2 and 3, the procedure as with Example 1 was performed except that the deposing treatment of polymer was carried out under the condition shown in Table 1. In Comparative Example 2, the amount of the deposited polymer was too much, whereas in Comparative Example 3, the amount of the deposited polymer was too little, thereby the dispersion stability of each case was found to be reduced.

### Comparative Example 4

The silane coupling agent treatment and the deposing treatment of polymer were omitted from the procedure of Example 1. That is, the magnetic particles themselves were used. In this case, the dispersion stability was very low, in which almost all particles had precipitated within a few minutes, so that the measurement according to DLS method could not be performed.

### Comparative Example 5

The silane coupling agent treatment and the deposing treatment of polymer were omitted from the procedure of Example 17. That is, the magnetic particles themselves were used. In this case, the dispersion stability was very low, in which almost all particles had precipitated within a few minutes, so that the measurement according to DLS method could not be performed.

TABLE 1

| | Raw material | | | | | | | | | | | | |
|---|---|---|---|---|---|---|---|---|---|---|---|---|---|
| | | | | | | | | Total | | | | | |
| | | | PEG chain | | | | | amount of | Physical features and properties of particle | | | | |
| | | | length | | | | | polymer | Primary | | Amount | | |
| | | | (Number | | | | | raw | particle | | of | | |
| | Acrylic acid | | of | PEG | | AMPS | | material | diameter | DLS | polymer | Dispersion | Magnetic |
| | [g] | [mmol] | PEG unit) | [mg] | [mmol] | [mg] | [mmol] | [g] | [nm] | [nm] | [wt %] | stability | collectivity |
| Example 1 | 0.68 | 9.5 | 9 | 37 | 0.15 | 70 | 0.34 | 0.79 | 24 | 154.3 | 15.4 | ⊚ | X |
| Example 2 | 0.68 | 9.5 | 9 | 74 | 0.15 | 35 | 0.17 | 0.79 | 24 | 164.1 | 16.6 | ⊚ | X |
| Example 3 | 0.68 | 9.5 | 9 | 37 | 0.07 | 35 | 0.17 | 0.75 | 24 | 174.0 | 17.1 | ⊚ | X |
| Example 4 | 0.68 | 9.5 | 9 | 37 | 0.07 | 70 | 0.34 | 0.79 | 24 | 128.9 | 17.0 | ⊚ | X |
| Example 5 | 0.68 | 9.5 | 9 | 74 | 0.15 | 0 | 0 | 0.75 | 24 | 167.1 | 16.8 | ⊚ | X |
| Example 6 | 0.68 | 9.5 | 9 | 74 | 0.15 | 0 | 0 | 0.75 | 24 | 180.4 | 17.0 | ⊚ | X |
| Example 7 | 1.1 | 14.6 | 9 | 53 | 0.11 | 0 | 0 | 1.15 | 24 | 194.0 | 17.1 | ⊚ | X |
| Example 8 | 0.89 | 12.4 | 9 | 53 | 0.11 | 0 | 0 | 0.94 | 24 | 163.0 | 17.1 | ⊚ | X |
| Example 9 | 0.74 | 10.2 | 9 | 37 | 0.07 | 0 | 0 | 0.78 | 24 | 125.8 | 14.8 | ⊚ | X |
| Example 10 | 0.63 | 8.7 | 9 | 30 | 0.06 | 0 | 0 | 0.66 | 24 | 171.2 | 14.4 | ⊚ | X |
| Example 11 | 0.95 | 13.1 | 4 | 45 | 0.16 | 0 | 0 | 1.00 | 24 | 97.2 | 10.2 | ⊚ | X |
| Example 12 | 0.74 | 10.2 | 4 | 35 | 0.13 | 0 | 0 | 0.78 | 24 | 109.1 | 10.6 | ⊚ | X |
| Example 13 | 1.6 | 21.9 | 4 | 75 | 0.27 | 0 | 0 | 1.68 | 24 | 141.3 | 10.6 | ⊚ | X |
| Example 14 | 1.3 | 17.5 | 4 | 60 | 0.22 | 0 | 0 | 1.36 | 24 | 146.8 | 10.4 | ⊚ | X |
| Example 15 | 0.74 | 10.2 | 14 | 35 | 0.13 | 0 | 0 | 0.78 | 24 | 139.6 | 13.8 | ⊚ | X |
| Example 16 | 0.68 | 9.5 | — | 0 | 0 | 35 | 0.17 | 0.72 | 24 | 108.2 | 12.5 | ⊚ | X |
| Example 17 | 0.68 | 9.5 | 9 | 35 | 0.07 | 35 | 0.17 | 0.75 | 230 | 346.4 | 2.2 | ◯ | ⊚ |
| Example 18 | 0.74 | 10.2 | 9 | 35 | 0.07 | 0 | 0 | 0.78 | 230 | 323.4 | 2.1 | ◯ | ⊚ |
| Example 19 | 0.68 | 9.5 | 9 | 35 | 0.07 | 70 | 0.34 | 0.79 | 230 | 266.3 | 2.2 | ◯ | ⊚ |
| Example 20 | 1.6 | 21.9 | 9 | 53 | 0.11 | 35 | 0 | 1.69 | 230 | 620.1 | 2.5 | ◯ | ⊚ |
| Comparative | 1.6 | 21.9 | — | 0 | 0 | 0 | 0 | 1.60 | 24 | 126.1 | 5.5 | X | ◯ |
| example 1 | | | | | | | | | | | | | |
| Comparative | 3.0 | 43.7 | 9 | 7 | 0.01 | 0 | 0 | 3.01 | 24 | 317.8 | 18.1 | X | ◯ |
| example 2 | | | | | | | | | | | | | |
| Comparative | 0.32 | 4.4 | 9 | 15 | 0.03 | 0 | 0 | 0.30 | 24 | 974.2 | 10.5 | X | ◯ |
| example 3 | | | | | | | | | | | | | |
| Comparative | — | — | — | — | — | — | — | | 24 | Unmeasur- | — | X | ◯ |
| example 4 | | | | | | | | | | able | | | |
| Comparative | — | — | — | — | — | — | — | | 230 | Unmeasur- | — | X | ◯ |
| example 5 | | | | | | | | | | able | | | |

Considering the fact that the dispersion stability was very low due to too much amount of the deposited polymer in Comparative Example 2 and too little amount of the deposited polymer in Comparative Example 3, it was suggested that the magnetic marker particles were suitably prepared using appropriate amount of polymer raw materials as shown in Examples 1 to 20; and also suggested that the suitable molar ratio among the carboxyl group and the polyethylene glycol chain and the sulfo group were those shown in Examples 1 to 20.

[symbol]

Evaluation of Dispersion Stability in pH Buffer Liquid

[symbol]

### (Evaluation of Stability by Visual Observation)

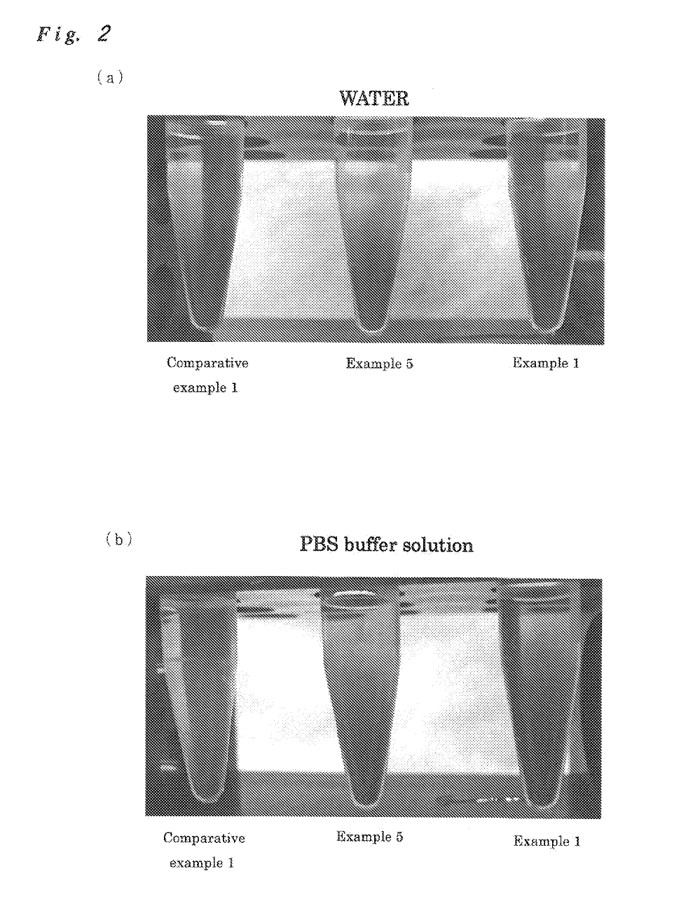

Using each of the particles obtained from Examples 1 and 5 and Comparative Example 1, the dispersion stability was evaluated. As the medium liquid, water and PBS buffer liquid were used. The concentration of the magnetic marker particles in the medium liquid was adjusted to be 1 mg/ml. The dispersion was left to stand for one month, thereafter the dispersion stability was evaluated based on the degree of its sedimentation. The results in the case of water medium are shown in FIG. 2(a) and the results in the case of PBS buffer liquid medium are shown in FIG. 2(b). In the case where the water was used, there was substantially little difference in the dispersion stability among Examples 1 and 5 and Comparative Example 1. However, the degree of the dispersion stability of the particles in the case of the PBS buffer liquid was shown as follows:

(Example 1)>(Example 5)>>>(Comparative Example 1).

Accordingly, it can be understood that the dispersion stability of the magnetic particles increases in the case where the deposited polymer further contained the sulfo group or polyethylene glycol chain, rather than the case where the deposited polymer contained only the carboxyl group.

### (Evaluation of Dispersion Stability Based on Zeta Potential)

It is presumed that the dispersion stability was provided by such a matter that the degree of the steric hindrance of the particles was increased by the crosslinked polymer chains via the polyethylene glycol chains (being condensable at both terminals), and that the zeta potential had increased by the existence of the sulfo group.

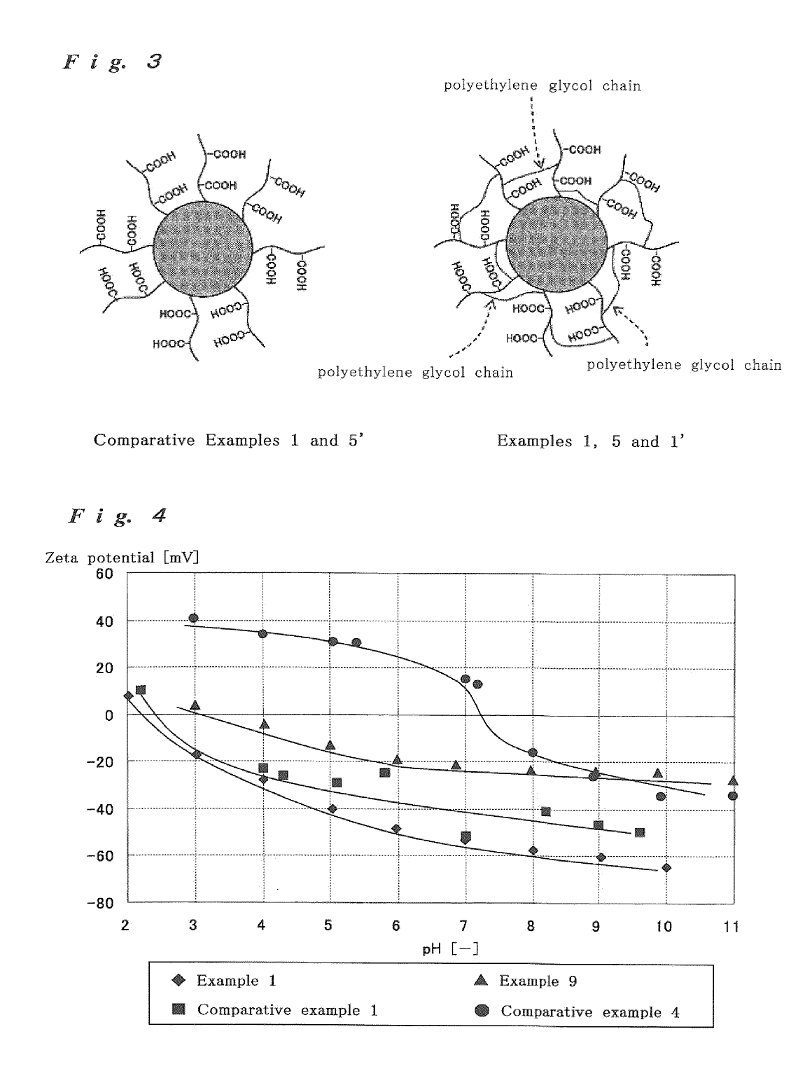

FIG. 3 shows schematic views of the crosslinked polymers. FIG. 4 shows results of the measurement of the zeta-potential. The zeta-potential was measured in each case, where the pH of the aqueous solution was varied by using hydrochloric acid and sodium hydroxide. As shown in FIG. 4, the relative amplitudes of the absolute value of the zeta-potential were as follows:

Example 1>Comparative Example 1>Example 9

The reason why the dispersion stability in Example 9 was higher than that of Comparative Example 1 despite that the zeta potential in Example 9 was lower than that of Comparative Example 1 may be considered that the degree of the steric hindrance of the particles was increased by the inclusion of the polyethylene glycol chains which had been condensable at both terminals, and that the ether chain moiety had high hydration force. With regard to the dispersion stability, Example 1 shows the best result among the above, wherein the high zeta potential and the increased steric hindrance are provided. Thus, it was found that the dispersion stability in pH buffer liquid was better in the case of the particles have a higher zeta potential and an increased steric hindrance as in the case of the magnetic marker particles of the present invention. Compared with Comparative Example 4, the zeta potential in the other cases broadly varies, which suggests that the surfaces of the core particles were surely coated with the polymer.

### (Evaluation of Dispersion Stability Based on Sedimentation Velocity)

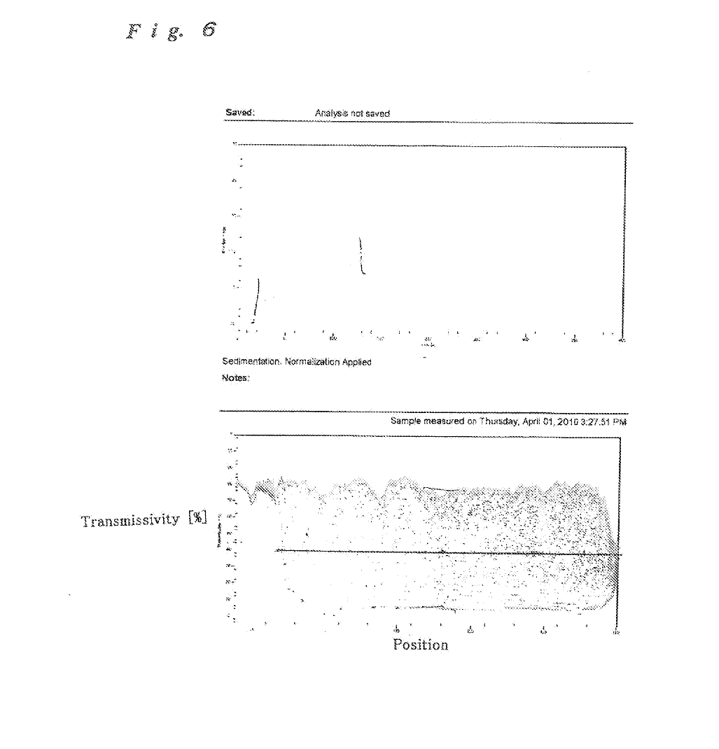

Sedimentation rates in phosphate buffered saline (PBS) and in water were measured using the particles obtained from Examples 1, 3, 7, 9, 12, 13, 17, 19 and 20 as well as Comparative Examples 4, 5. As the measurement device, LUMiFuge 110 (manufactured by Nihon RUFUTO) was used. As the measurement condition, the speed of rotation was 2000 rpm and the centrifugal force was 525×g in the measurement using PBS in Examples 17, 19 and 20. While on the other hand, the speed of rotation was 500 rpm and the centrifugal force was 35×g in the measurement using water. Further, the speed of rotation was 200 rpm and the centrifugal force was 5×g in the measurement using PBS in Comparative Examples 4 and 5. Furthermore, the speed of rotation was 200 rpm and the centrifugal force was 5×g in the measurement using water. In the other Examples and Comparative Examples, the speed of rotation was 4000 rpm in the measurement using PBS as well as water. In this case, the centrifugal force was 2300×g. As such, the speed of rotation and thus the centrifugal force were able to be optionally set as necessary. The sample to be tested was introduced into the device and the transmission factor (transmissivity) was measured. After the measurement, the value of sedimentation velocity V_{S} was calculated based on the positional variation in the sample cell using the initial transmission factor when the sample was set and a medium value of the transmission factor at the completion of the measurement (with regard to the example of the raw data for these calculation, see FIG. 6). The raw data of FIG. 6 were obtained from LUMiFuge (manufactured by LUM). Thereafter, the obtained value V_{S} was divided by the centrifugal force so as to eliminate the influence of the centrifugal force, and thereby obtaining the sedimentation velocity according to the present invention. That is, the sedimentation velocity V_{B} was calculated based on the above-mentioned Formula 1. Then, a ratio of the sedimentation velocities V in PBS to that in water was obtained (that is, the ratio V_{B}/V_{W} was evaluated). Furthermore, the sedimentation velocity V′ was obtained by dividing the sedimentation velocity V_{B} with square of the primary particle diameter. These results are shown in Table 2.

TABLE 2

| | | Dispersion Medium: Water | | | | Dispersion Medium: PBS | | | | |
|---|---|---|---|---|---|---|---|---|---|---|
| | Primary | Centri- | Sedimentation | Sedi- | Sedi- | Centri- | Sedimentation | Sedi- | Sedi- | Ratio of |
| | particle | fugal | velocity | mentation | mentation | fugal | velocity | mentation | mentation | Sedimentation |
| | diameter | force | Vs | velocity | velocity | force | Vs | velocity | velocity | velocity |
| | (nm) | (xg) | (μm/s) | V_{W} | V′ | (xg) | (μm/s) | V_{B} | V′ | V_{B}/V_{W} |
| Example 1 | 24 | 2300 | 19 | 8.3 × 10⁻³ | 1.43 × 10⁻⁵ | 2300 | 20 | 8.7 × 10⁻³ | 1.51 × 10⁻⁵ | 1.1 |
| Example 3 | 24 | 2300 | 16 | 7.0 × 10⁻³ | 1.21 × 10⁻⁵ | 2300 | 18 | 7.8 × 10⁻³ | 1.36 × 10⁻⁵ | 1.1 |
| Example 7 | 24 | 2300 | 34 | 1.5 × 10⁻² | 2.57 × 10⁻⁵ | 2300 | 35 | 1.5 × 10⁻² | 2.64 × 10⁻⁵ | 1.0 |
| Example 9 | 24 | 2300 | 13 | 5.7 × 10⁻³ | 9.81 × 10⁻⁶ | 2300 | 14 | 6.1 × 10⁻³ | 1.06 × 10⁻⁵ | 1.1 |
| Example 12 | 24 | 2300 | 23 | 1.0 × 10⁻² | 1.74 × 10⁻⁵ | 2300 | 26 | 1.1 × 10⁻² | 1.96 × 10⁻⁵ | 1.1 |
| Example 13 | 24 | 2300 | 46 | 2.0 × 10⁻² | 3.47 × 10⁻⁵ | 2300 | 48 | 2.1 × 10⁻² | 3.62 × 10⁻⁵ | 1.0 |
| Example 17 | 230 | 525 | 65 | 1.2 × 10⁻¹ | 2.34 × 10⁻⁶ | 35 | 70 | 2.0 | 3.78 × 10⁻⁵ | 16.2 |
| Example 19 | 230 | 525 | 50 | 9.5 × 10⁻² | 1.80 × 10⁻⁸ | 35 | 54 | 1.5 | 2.92 × 10⁻⁵ | 16.2 |
| Example 20 | 230 | 525 | 133 | 2.5 × 10⁻¹ | 4.79 × 10⁻⁶ | 35 | 140 | 4.0 | 7.56 × 10⁻⁵ | 15.8 |
| Comparative | 24 | 5 | 291 | 58.2 | 1.01 × 10⁻¹ | 5 | 294 | 58.8 | 1.02 × 10⁻¹ | 1.0 |
| Example 4 | | | | | | | | | | |
| Comparative | 230 | 5 | 242 | 48.4 | 9.15 × 10⁻⁴ | 5 | 284 | 56.8 | 1.07 × 10⁻³ | 1.2 |
| Example 5 | | | | | | | | | | |

With reference to Table 2, it was found that each of the particles showed high dispersion stability in water. It was also found that, with regard to the PBS, the values of V_{B} and V′ were generally low in Examples, and consequently the dispersion stabilities thereof were high (for example, the value of V_{B} in the case of Examples 1, 3, 7, 9, 12 and 13 was in the range of 6.1×10⁻³ to 2.1×10⁻² and the value of V_{B} in the case of Examples 17 to 19 was in the range of 1.5 to 4.0). Further, it was found that, the values of V_{B} and V′ in the case of Comparative Examples 4 and 5 were higher than those of Examples, and consequently the dispersion stabilities of the case of Comparative Examples 4 and 5 were low. Thus, it can be understood that the magnetic marker particles of the present invention have high dispersion stabilities even in the PBS.

[symbol]

Evaluation of Magnetic Collectivity

[symbol]

Magnetic collection rates were measured in a phosphate buffered saline (PBS) and in water with respect to the particles obtained from Example 17 and the particles Dynabeads (MyOne Carboxylic acid (manufactured by Invitrogen Corporation)) as Comparative Example. As the measuring device, bio-spectrophotometer U-0080D (manufactured by Hitachi High-Technologies Corporation) was used. Specifically, a dispersion liquid of the magnetic particles (0.2 mg/mL) was introduced into a spectroscopic cell having 1 cm×1 cm square bottom, and the cell was placed in a spectrophotometer. After the particles were sufficiently dispersed by pipetting, a neodymium magnet NK037 (manufactured by Niroku Seisakusho) (outer size: 40 mm×20 mm×1 mm, surface magnetic flux density: 134 mT) was brought closer to the outside of the cell and then measured the variation of the light absorbance at 550 nm with time. The magnetic field inside of the cell in this case was measured by the above-mentioned method. As a result, the value of the magnetic field was 0.36 T.

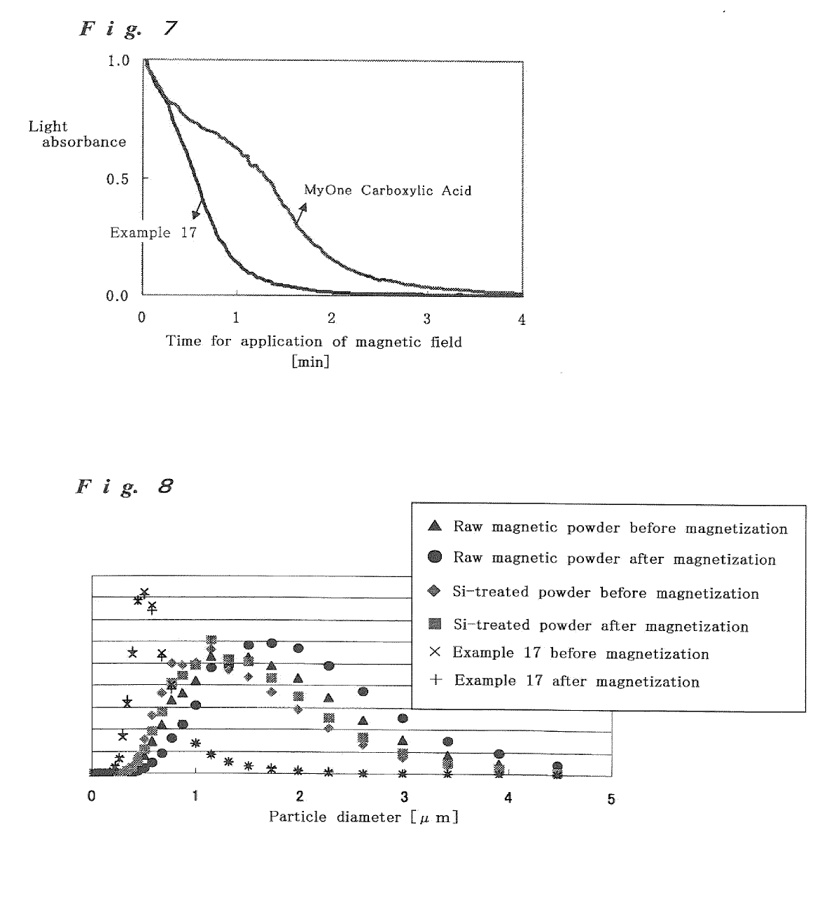

FIG. 7 shows the results of the measurement. As seen from FIG. 7, the light absorbance had decreased in a short period of time in Example 17. Specifically, the relative light absorbance of the buffer solution in the case of Example 17 had decreased from its initial value “1” to about 0.15 in about 60 seconds after the application of the magnetic field. That is, it was found that the magnetic marker particles of the present invention could be effectively magnetically collected in a shorter period of time in the dispersion of the particles-containing buffer solution.

[symbol]

Evaluation of Re-Dispersibility

[symbol]

Evaluation tests were carried out in order to confirm the effects of “re-dispersibility (i.e. dispersibility or dispersion stability after the magnetic collection)” Specifically, each particles of “Example 17”, “the raw material powder of Example 17 (i.e., raw magnetic powder of Comparative Example 5)” and “particles obtained by subjecting the raw material powder of Example 17 to the silane coupling agent treatment (i.e. Si treated powder)” were dispersed in each solution of the phosphate buffered saline (PBS) (10 mg/ml), respectively. Each of the resultant buffer dispersions was subjected to the operation composed of “particles aggregation due to the magnetic collection” and “re-dispersion by using of microwave” at the following conditions, which operations was repeated ten times:
Magnetic collection operation: an operation of applying a magnetic field of 0.24 T to the whole buffer solution for 2 minutes (using a stand for separating magnetic beads “Magical Trapper” (manufactured by Toyobo Co., Ltd.), magnetic field measurement apparatus: “Handy Teslameter Elulu DTM6100” (manufactured by Mytech Corporation);
Ultrasonic irradiation operation (re-dispersion operation): an operation of applying ultrasonic energy to the “area of the aggregated magnetic marker particles” for 2 minutes using an ultrasonic cleaner (VS-150, frequency 50 kHz, output 150 W) (manufactured by As-One Corp.).

Before and after the above operations, the dispersion particle diameter (i.e., secondary particle diameter) was measured, and thereby the degrees of the magnetic aggregation were compared. For the above measurement of the dispersion particle diameter, a laser diffraction/scattering particle size distribution analyzer LA-920 (manufactured by Horiba Ltd.) was used. It should be noted that the measurement of the above dispersion particle diameter was carried out using the DLS method, which was different from this laser diffraction/scattering particle size distribution analyzer LA-920 (manufactured by Horiba Ltd.). The reason for this is that the measurable range is in the range of a few nm to 5 μm in the DLS method, thus DLS method is considered not to be suitable for measuring the degree of the magnetic aggregation (since their measurement principles differ from each other, it often happens that different results are obtained depending on kinds of the measuring methods even if the same particles are used.).

The results of “evaluation of re-dispersibility” are shown in Table 3 and FIG. 8. In Table 3 and FIG. 8, respective particles of “Example 17”, “the raw material powder (Comparative Example 5)” and “Si treated powder” were compared with each other and thus evaluated. The standard deviation of the particle diameters expresses the width of the particle size distribution, wherein the larger standard deviation indicates broader particle size distribution. It was found that both of the raw material powder (Comparative Example 5) and the Si treated powder had large average particle diameters and large particle size distributions before the magnetic collection, so that they had already formed broad aggregations and each of them tended to easily aggregate. On the other hand, Example 17 had narrow average particle diameters and narrow particle size distributions, so that it was found that the particles had fewer aggregations and tended to hardly aggregate. With regard to the distributions between before and after magnetization, those of Example 17 did not change, in contrast, those of “raw magnetic powder (Comparative Example 5)” and “Si treated powder” had enlarged. In addition, the dispersion particle diameter had substantially no change in Example 17, in contrast, those of the raw magnetic powder (Comparative Example 5) had enlarged by about 20%, and those of the Si treated powder had enlarged by about 10%. That is, in the cases of the raw magnetic powder (Comparative Example 5) and the Si treated powder, the particles had originally tended to easily aggregate and formed a broad aggregations, and furthermore the magnetic aggregations thereof had been promoted due to the magnetic field.

According to the above results, the magnetic marker particles of the present invention exhibited favorable re-dispersibility. These results seem to be due to the matter that the polymer, which coated the surface of the present particles, had a high steric hindrance effect. That is, it is conceivable that the force for suppressing the aggregation of the particles was larger than the force for forming the aggregation of the particles, and thereby an effect to effectively suppress the aggregation was exerted.

TABLE 3

| | | Diameter | Increase | |
|---|---|---|---|---|
| | | of | rate of | |
| | | dispersed | diameter | |
| | | particles | of | Primary |
| | Magnetization | (avarage | dispersed | particle |
| | (performed ten | diameter) | particles | diameter |
| Sample | times) | [μm] | (%) | [μm] |
| Example 17 | Before | 0.54 ± 0.23 | 3.7 | 0.23 |
| | magnetization | | | |
| | After | 0.56 ± 0.23 | | |
| | magnetization | | | |
| Raw magnetic | Before | 1.41 ± 0.69 | 19.1 | 0.23 |
| powder | magnetization | | | |
| (Comparative | After | 1.68 ± 0.76 | | |
| example 5) | magnetization | | | |
| Si-treated | Before | 1.16 ± 0.58 | 8.6 | 0.23 |
| powder | magnetization | | | |
| | After | 1.26 ± 0.6 | | |
| | magnetization | | | |

[symbol]

Immobilization Test of Biomaterial-Binding Material

[symbol]

Avidin was immobilized on the magnetic marker particles of Examples 1 and 9 and Comparative Example 1. Specifically, in each of Examples 1 and 9 and Comparative Example 1, the polymer coated magnetic particles obtained therefrom (each 2 mg) were dispersed in 1 ml of 25 mM MES buffer liquid to form 1 ml of polymer coated magnetic particles liquid. Then, to the obtained polymer coated magnetic particles liquid, “0.5 ml of solution, in which 5 mg of EDC was dissolved in 0.5 ml of 25 mM MES buffer liquid (pH 6.0)” and “0.5 ml of solution, in which 5 mg of Sulfo-NHS was dissolved in 0.5 ml of 25 mM MES buffer liquid (pH 6.0)” were added to form 2 ml volume of liquid, thereafter the resulting liquid was stirred for 15 minutes. After filtrating it by a spin column, 1 ml of 25 mM MES buffer liquid (pH 6.0) was further added and the resultant liquid was filtered and washed, and then the polymer coated magnetic particles were dispersed in 10 mM phosphate buffer liquid (pH 8.3) to obtain 1 ml of liquid thereof.

Then, 1 mg of streptavidin (manufactured by Wako Pure Chemical Industries, Ltd.) was dissolved in 0.5 ml of 10 mM phosphate buffer liquid (pH 8.3), to which 0.5 ml of polymer coated magnetic particles liquid was added and then supersonic was applied to the liquid for 1 hour. Then, the liquid was stirred by tube mixer for further one hour. Then, after being filtered by the spin column, 1 ml of 10 mM phosphate buffer liquid (pH 7.2) was added to the liquid, which was filtered and washed by the spin column for 5 times. As a result, there was obtained polymer coated magnetic particles on which streptavidin were immobilized. Finally, the streptavidin-immobilized polymer coated magnetic particles were recovered by 10 mM phosphate buffer liquid (pH 7.2) to obtain 1 ml of liquid thereof.

### (Evaluation Test of Specific Binding Ability)

In order to evaluate the specific binding ability between the streptavidin-immobilized polymer coated magnetic particles and biotin, the biotin-bound amount of the streptavidin-immobilized polymer coated magnetic particles was evaluated by using biotin-fluorescein (manufactured by PIERCE).

First, in each of Examples 1 and 9 and Comparative Example 1, the polymer coated magnetic particles obtained therefrom were streptavidin-immobilized. The streptavidin-immobilized polymer coated magnetic particles were dispersed in 0.05 mg/ml of PBS buffer liquid, from which each quantity of sample of 0 μl, 10 μl, 50 μl, 100 μl, 250 μl was taken and each introduced to separate Eppendorf tubes, respectively. Next, a series of dilutions in which the total volume was 250 μl were prepared by adding PBS buffer liquid and then 500 μl of 40 nM biotin-fluorescein solution dissolved in PBS buffer liquid was added to each sample to obtain 750 μl of liquid. Next, the liquid was stirred at 1500 rpm using a tube mixer for 10 minutes, followed by being subjected to the magnetic separation for 20 minutes. After the magnetic separation was performed, 500 μl of the supernatant liquid was subjected to a centrifugal treatment at 28700×g for 10 minutes. From the resultant liquid, 100 μl of supernatant liquid was taken and added to a microplate, which was observed by a microplate reader (infinite F200 (manufactured by TECAN)) using 485 nm of excitation wavelength and 535 nm of fluorescent wavelength. Thereby, the biotin-bound amount of the streptavidin-immobilized polymer coated magnetic particles was evaluated from the fluorescence drop of biotin-fluorescein. The results are shown in Table 4.

TABLE 4

| | Bound amount of biotin-fluorescein |
|---|---|
| | [mol/mg] |
| Example 1 | 6.5 × 10⁻¹⁰ |
| Example 9 | 5.9 × 10⁻¹⁰ |
| Comparative | 3.0 × 10⁻¹⁰ |
| example 1 | |

### (Evaluation Test of Nonspecific Binding Ability)

In light of the fact that the binding between the biotin-fluorescein and the streptavidin-immobilized polymer coated magnetic particles can be a nonspecific binding, the nonspecific binding ability was evaluated by using uranine (manufactured by Wako Pure Chemical Industries, Ltd.) corresponding to the fluorescent moiety of the biotin-fluorescein.

First, in each of Examples 1 and 9 and Comparative Example 1, the polymer coated magnetic particles obtained therefrom were subjected to a streptavidin-immobilization treatment. The streptavidin-immobilized polymer coated magnetic particles were then dispersed in 0.05 mg/ml of PBS buffer liquid, from which each quantity of sample of 0 μl, 10 μl, 50 μl, 100 μl, 250 μl was taken and each introduced to separate Eppendorf tubes, respectively. Next, a series of dilutions in which the total volume was 250 μl were prepared by adding PBS buffer liquid and then 500 μl of 40 nM uranine solution dissolved in PBS buffer liquid was added to each sample to obtain 750 μl of liquid. Then, the liquid was stirred at 1500 rpm using a tube mixer for 10 minutes, followed by being subjected to the magnetic separation for 20 minutes. After the magnetic separation was performed, 500 μl of the supernatant liquid was subjected to a centrifugal treatment at 28700×g for 10 minutes. From the resultant liquid, 100 μl of supernatant liquid was taken and added to a microplate, which was observed by a microplate reader using 485 nm of excitation wavelength and 535 nm of fluorescent wavelength. As a result, the uranine-bound amount of the nonspecific binding regarding the streptavidin-immobilized polymer coated magnetic particles was evaluated from the fluorescence drop of uranine. The results are shown in Table 5.

TABLE 5

| | Bound amount of uranine |
|---|---|
| | [mol/mg] |
| Example 1 | 0 |
| Example 9 | 3.0 × 10⁻¹³ |
| Comparative | 0 |
| example 1 | |

According to the results shown in Tables 4 and 5, it was confirmed that the bound amount of the biotin-fluorescein was larger than the bound amount of uranine, so that the streptavidin-immobilized polymer coated magnetic particles were capable of specifically binding to the biotin. That is, it can be understood that the magnetic marker particles of the present invention are suitably available as a marker used in the biotechnological field or life-science field.

### “Case (B): Specialized in the Magnetic Marker Particles Each Having a Spherical Shape”

[symbol] Preparation of Particles

[symbol]

As Examples and Comparative Examples relating to the magnetic marker particles each having a spherical shape, the following particles were prepared:

### Example 1′

### Synthesis of Magnetite Particles

As the reaction system, the anaerobic condition was adopted. Water and glycerin were deaerated using nitrogen gas. During the reaction, the reactor was replaced with nitrogen gas, thereby no oxygen-condition was formed. The nitrogen gas with its purity of 99.998% was used.

Magnetite particles serving as the core particles were synthesized according to the procedures as follows:

First, 1.1 g ferrous sulfate (FeSO₄.7H₂O) was dissolved in 4 cc pure water to form an aqueous solution of ferrous sulfate. The resultant ferrous sulfate was mixed with 120 cc of glycerin to form a uniform solution. Apart from this, 112 g of sodium hydroxide was dissolved in 100 cc of pure water to form an aqueous solution of sodium hydroxide. Next, 14.7 cc of aqueous solution of sodium hydroxide was added dropwise to the aqueous solution of ferrous sulfate while stirring the ferrous sulfate solution to form a precipitation of ferrous hydroxide. Water was added dropwise so as to adjust the final volume to be 145 cc. After this adding of water, the solution was stirred for 30 minutes. The resultant solution was introduced in a pressure-tight reactor and then reacted for 20 hours at a temperature of 180° C. by a dryer. The resultant particles were washed and then used for the next reaction without being dried. As a result, the resultant magnetite particles had almost spherical shapes having the ratio of the largest radius to the smallest radius of 1.14 and also had a primary particle diameter of 250 nm (the ratio of the largest radius to the smallest radius and the primary particle diameter of the magnetite particles were obtained as a number average of 300 particles after measuring each size thereof from a micrograph of transmission-type electron microscope using an image analyzing software Image-Pro Plus (manufactured by Nippon Roper Co., Ltd.) The magnetite particles had a saturation magnetization of 77.6 A·m²/kg (emu/g) and a coercive force of 3.10 kA/m (38.9 oersteds).

### Examples 2′ to 7′

The procedure as with Example 1′ was performed except that the magnetite particles were prepared under the condition as shown in Table 6. The results of the measured particle diameter and magnetic properties are summarized in Table 7.

### Example 8′

The procedure as with Example 1′ was performed except that the magnetite particles were prepared under the condition as shown in Table 6 and the microwave irradiation was adopted as the heating treatment of the magnetite particles preparation. The results of the measured particle diameter and magnetic properties are summarized in Table 7. As the heating device for the microwave irradiation, MicroSYNTH (manufactured by Milestone general company) was used.

### Example 9′

The procedure as with Example 1′ was performed except that the procedure was carried out under the aerobic condition instead of the anaerobic condition. The results of the measured particle diameter and magnetic properties are summarized in Table 7.

### Comparative Examples 1′ to 4′

The procedure as with Example 1′ was performed except that the magnetite particles were prepared under the condition as shown in Table 6. The results of the measured particle diameter and magnetic properties are summarized in Table 7. Comparative Examples 1′ to 4′ were intended so as to prepare the particles having non-spherical shapes relative to the above Examples 1′ to 7′ by varying the amount of alkali or reaction time period. In this regard, it was confirmed that the particles each having a spherical shape could not be obtained in the case where the amount of alkali was different even when the reaction time period was the same, or in the case where the reaction time period was different even when the amount of alkali was the same.

### Comparative Examples 5′ and 6′

The commercially available magnetite particles TM-023 (manufactured by Toda Kogyo KK) were used. The particles had a primary particle diameter of 230 nm, CV of 22.0, radius ratio of 1.46 (i.e., ratio of the largest radius to the smallest radius being 1.46), and were compose of particles with their shape being cubic having rounded corners and with their shape being irregular shape.

TABLE 6

| | | | Amount of | Amount of | | Reaction | Reaction |
|---|---|---|---|---|---|---|---|
| | Source of | Source of | alkali | glycerin | Source | temperature | time |
| | Fe ion | alkali | (mol) | (mL) | of heat | [° C.] | [h] |
| Example 1′ | FeSO₄ | KOH | 0.2 | 120 | dryer | 180 | 20 |
| Example 2′ | FeSO₄ | KOH | 0.23 | 120 | dryer | 180 | 20 |
| Example 3′ | FeSO₄ | KOH | 0.17 | 120 | dryer | 180 | 20 |
| Example 4′ | FeSO₄ | KOH | 0.06 | 100 | dryer | 180 | 20 |
| Example 5′ | FeSO₄ | KOH | 0.1 | 120 | dryer | 180 | 20 |
| Example 6′ | FeSO₄ | KOH | 0.12 | 120 | dryer | 200 | 20 |
| Example 7′ | FeSO₄ | KOH | 0.2 | 120 | dryer | 180 | 10 |
| Example 8′ | FeSO₄ | KOH | 0.3 | 120 | microwave | 200 | 10 |
| Example 9′ | FeSO₄ | KOH | 0.2 | 120 | dryer | 180 | 20 |
| Comparative | FeSO₄ | KOH | 0.05 | 120 | dryer | 180 | 20 |
| example 1′ | | | | | | | |
| Comparative | FeSO₄ | KOH | 0.5 | 120 | dryer | 180 | 20 |
| example 2′ | | | | | | | |
| Comparative | FeSO₄ | KOH | 0.2 | 120 | dryer | 180 | 5 |
| example 3′ | | | | | | | |
| Comparative | FeSO₄ | KOH | 0.2 | 120 | dryer | 180 | 40 |
| example 4′ | | | | | | | |

TABLE 7

| | | | Ratio of | | |
|---|---|---|---|---|---|
| | Primary | | largest | | |
| | particle | | diameter | Saturation | Coercive |
| | diameter | CV | to smallest | magnetization | force |
| | (nm) | (%) | diameter | (A · m²/kg) | (kA/m) |
| Example 1′ | 250 | 12.6 | 1.14 | 77.6 | 3.10 |
| Example 2′ | 235 | 15.6 | 1.20 | 76.8 | 3.78 |
| Example 3′ | 240 | 15.9 | 1.19 | 76.3 | 3.67 |
| Example 4′ | 510 | 16.5 | 1.22 | 78.9 | 3.98 |
| Example 5′ | 140 | 12.7 | 1.17 | 71.5 | 3.23 |
| Example 6′ | 270 | 16.3 | 1.16 | 77.9 | 3.18 |
| Example 7′ | 240 | 11.6 | 1.13 | 76.6 | 3.08 |
| Example 8′ | 240 | 10.6 | 1.11 | 78.1 | 3.08 |
| Example 9′ | 260 | 13.1 | 1.16 | 79.6 | 3.12 |
| Comparative | 24 | 21.1 | 1.41 | 68.6 | 5.20 |
| example 1′ | | | | | |
| Comparative | 560 | 23.1 | 1.53 | 79.1 | 5.83 |
| example 2′ | | | | | |
| Compartive | 24 | 19.5 | 1.51 | 69.5 | 5.14 |
| example 3′ | | | | | |
| Comparative | 270 | 23.4 | 1.35 | 75.4 | 4.56 |
| example 4′ | | | | | |
| Comparative | 250 | 22.0 | 1.46 | 83.7 | 5.22 |
| example | | | | | |
| 5′, 6′ | | | | | |

It was found that the magnetite particles of Examples 1′ to 9′ had smaller ratios between the long axis and short axis (i.e., smaller ratios of the largest radius to the smallest radius) and smaller CV values compared with the particles of Comparative Examples 1′ to 6′, so that they were well ordered in terms of shapes. With regard to the ratios of the largest radius to the smallest radius, the ratios of Examples 1′ to 7′ were in the range of 1.1 to 1.25, whereas the ratios of Comparative Examples 1′ to 6′ were in the range of 1.4 to 1.6. That is, the particles obtained from Examples 1′ to 9′ had substantially spherical shapes. In addition, the particles obtained from Examples 1′ to 9′ had smaller coercive force. Such smaller coercive force seemed to be due to small geometric magnetic anisotropies of the particles, caused by the spherical shape thereof.

The particles obtained from the above Examples and Comparative Examples were subjected to “Silane coupling agent treatment” and “Depositing treatment of polymer” as described infra.

### Examples 1′ to 9′

### Silane Coupling Agent Treatment

The magnetite particles obtained from the above reaction (200 mg) were dispersed in 50 ml of methanol. To this dispersion liquid, 3 ml of 3-methacryloxypropyl trimethoxysilane (LS-3360, manufactured by Shin-Etsu Chemical Co., Ltd.) was added and stirred at 40° C. for 4 hours. Subsequently, the resulting suspension was subjected to a centrifugal treatment and washed, and then the solvent medium was replaced with water. As a result, there was obtained the magnetic particles with the silane coupling agent deposited on the surface thereof.

### <Depositing Treatment of Polymer>

200 mg of the magnetic particles to which the silane coupling agent deposited were dispersed in 50 ml of water. The resultant dispersion was stirred while blowing nitrogen gas thereinto so as to prepare a nitrogen atmosphere. Thereafter, 0.68 g of acrylic acid (manufactured by Wako Pure Chemical Industries, Ltd.), 35 μl of Light-Acrylate 9EG-A (hereinafter referred to also as “PEG”) (manufactured by KYOEISHA CHEMICAL Co., LTD.), 35 mg of 2-acrylamido-2-methylpropanesulfonic acid (hereinafter referred to also as “AMPS”) (manufactured by Wako Pure Chemical Industries, Ltd.) were added to the dispersion. While stirring the dispersion for a while, 1.4 mg of 2,2′-azobis(2-methylpropionamidine)dihydrochloride (manufactured by Wako Pure Chemical Industries, Ltd.) was added thereto and reacted under the nitrogen atmosphere at 70° C. for 4 hours. Then, the particles were washed by using the centrifugation technique. As a result, the magnetic marker particles with the deposited polymer thereon were obtained. The particle size of these particles was calculated based on the electron microscope micrograph so as to obtain “ratio of the largest radius to the smallest radius” and “primary particle diameter”. The ratio of the largest radius to the smallest radius was 1.14 and the primary particle diameter was 250 nm*¹. As a result, it was understood that not only the core particles had the spherical shapes, but also the marker particles, even though they were those obtained after the depositing treatment of polymer, also had the spherical shapes. *¹ Although the alteration of the particle diameter before and after the polymer deposition could not be observed, it seemed to be caused by the performance of the electron microscope used for the observation. Specifically, the observation was carried out using the transmission-type electron microscope (TEM), however the electron beam of TEM was easily transmitted through the light elements (e.g., carbon, nitrogen), and the polymer layer itself was in an invisible state, which could be a factor thereof.

### <Measurement of Dispersion Particle Diameter and Amount of Deposited Polymer>

Together with measuring the amount of the deposited polymer, the dispersion particle diameter was measured according to DLS method by dispersing the magnetic marker particles in the buffer liquid. The measurement of the amount of deposited polymer was performed according to the thermogravimetric method after the magnetic marker particles were dried. Specifically, the amount of deposited polymer was measured from the loss in weight of the particles upon combustion of the organic materials (polymer) using a thermogravimetric analyzer TG-DTA 2000S (manufactured by Macscience). As a result, the amount of deposited polymer was 2.5% by weight and the dispersion particle diameter was 297 nm.

### Examples 10′ to 15′

The procedures as with Examples 1′ to 7′ were performed except that the depositing treatment of polymer was performed under the condition as shown in Table 8 infra.

### Examples 16′ to 19′

Instead of Light-Acrylate 9EG-A (manufactured by KYOEISHA CHEMICAL Co., LTD.), Light-Acrylate 4EG-A (manufactured by KYOEISHA CHEMICAL Co., LTD.) having different length of polyethylene glycol chain was used. Since this Light-Acrylate 4EG-A has low solubility in water, the procedure was carried out in a mixture solvent of water and methanol. Except these, the procedure was carried out as with those of Examples 1′ to 7′. The conditions used in these procedures are shown in Table 8 infra.

### Example 20

Instead of Light-Acrylate 9EG-A (manufactured by KYOEISHA CHEMICAL Co., LTD.), Light-Acrylate 14EG-A (manufactured by KYOEISHA CHEMICAL Co., LTD.) having different length of polyethylene glycol chain was used. Except this, the procedure was carried out as with those of Examples 1′ to 7′. The conditions used in these procedures are shown in Table 8 infra.

### Example 21′

The procedures as with Examples 1′ to 7′ were performed except that Light-Acrylate 9EG-A was not used. The conditions used in these procedures are shown in Table 8 infra.

### Examples 22′ to 25′

The procedures as with Examples 1′ to 7′ were performed except that the monomers were changed to use acrylic acid-2-hydroxylethyl (HEA) (manufactured by Wako Pure Chemical Industries, Ltd.) having a hydroxyl group therein, HOA-MS (manufactured by KYOEISHA CHEMICAL Co., LTD.) having a carboxyl group therein and Light-Acrylate 9EG-A (manufactured by KYOEISHA CHEMICAL Co., LTD.) having PEG therein. The conditions used in these procedures are shown in Table 9 infra.

### Example 26′

The procedures as with Example 20′ was performed except that 2-acrylamido-2-methylpropanesulfonic acid (manufactured by Wako Pure Chemical Industries, Ltd.) as the monomer having a sulfone group was additionally used. The conditions used in these procedures are shown in Table 9 infra.

### Comparative Example 5′

The procedure as with Example 1′ was performed except that the deposited polymer formation treatment was performed using only the 1.6 g of acrylic acid, not using Light-Acrylate 9EG-A and 2-acrylamido-2-methylpropanesulfonic acid. The conditions used in these procedures are shown in Table 8 infra.

### Comparative Examples 6′ and 7′

The procedure as with Example 1′ was performed except that the depositing treatment of polymer was performed under the condition as shown in Table 8 infra. The conditions used in these procedures are shown in Table 8 infra. In Comparative Example 6′, the amount of the deposited polymer was too much, and in Comparative Example 7′, the amount of the deposited polymer was too little, and consequently the dispersion stability of each case was found to be reduced.

### Comparative Example 8′

The silane coupling agent treatment and the depositing treatment of polymer were omitted from the procedure of Example 1′. That is, the magnetic particles themselves were used. The conditions used in these procedures are shown in Table 8 infra. In this case, the dispersion stability was very low, in which almost all particles had precipitated within a few minutes, so that the measurement according to DLS method could not be performed.

### Comparative Examples 9′ and 10′

The procedure as with Example 1′ was performed except that Magnetite TM-023 (manufactured by Toda Kogyo K.K.) (primary particle diameter: 230 nm) was used as the core particle and the amount of the monomer was changed as shown in Table 8. The conditions used in these procedures are shown in Table 8 infra.

TABLE 8

| | Acrylic | PEG | | | | | Amount of |
|---|---|---|---|---|---|---|---|
| | acid | chain | PEG | AMPS | Primary particle | DLS | polymer |
| | (mL) | length | (μL) | (mg) | diameter (nm) | (nm) | (wt %) |
| Example 1′ | 0.65 | 9 | 35 | 35 | 250 | 297 | 2.5 |
| Example 2′ | 0.65 | 9 | 35 | 35 | 235 | 285 | 2.4 |
| Example 3′ | 0.65 | 9 | 35 | 35 | 240 | 290 | 2.5 |
| Example 4′ | 0.65 | 9 | 35 | 35 | 510 | 671 | 2.3 |
| Example 5′ | 0.65 | 9 | 35 | 35 | 140 | 213 | 2.0 |
| Example 6′ | 0.65 | 9 | 35 | 35 | 270 | 325 | 2.4 |
| Example 7′ | 0.65 | 9 | 35 | 35 | 240 | 289 | 2.4 |
| Example 8′ | 0.65 | 9 | 35 | 35 | 240 | 280 | 2.3 |
| Example 9′ | 0.65 | 9 | 35 | 35 | 260 | 294 | 2.5 |
| Example 10′ | 0.65 | 9 | 70 | 35 | 250 | 352 | 2.5 |
| Example 11′ | 0.65 | 9 | 35 | 70 | 250 | 330 | 2.6 |
| Example 12′ | 0.65 | 9 | 70 | 0 | 250 | 370 | 2.5 |
| Example 13′ | 1 | 9 | 50 | 0 | 250 | 376 | 2.7 |
| Example 14′ | 0.7 | 9 | 35 | 0 | 250 | 294 | 2.4 |
| Example 15′ | 0.6 | 9 | 30 | 0 | 250 | 302 | 2.4 |
| Example 16′ | 0.9 | 4 | 45 | 0 | 250 | 281 | 2.3 |
| Example 17′ | 0.7 | 4 | 35 | 0 | 250 | 286 | 2.4 |
| Example 18′ | 1.5 | 4 | 75 | 0 | 250 | 343 | 2.7 |
| Example 19′ | 1.2 | 4 | 60 | 0 | 250 | 339 | 2.6 |
| Example 20′ | 0.7 | 14 | 35 | 0 | 250 | 291 | 2.6 |
| Example 21′ | 0.65 | — | 0 | 35 | 250 | 305 | 2.3 |
| Comparative | 1.5 | — | 0 | 0 | 250 | 364 | 2.5 |
| example 5′ | | | | | | | |
| Comparative | 3 | 9 | 7 | 0 | 250 | 563 | 5.1 |
| example 6′ | | | | | | | |
| Comparative | 0.3 | 9 | 15 | 0 | 250 | 981 | 0.8 |
| example 7′ | | | | | | | |
| Comparative | — | — | — | — | 250 | — | 0 |
| example 8′ | | | | | | | |
| Comparative | 0.65 | 9 | 35 | 35 | 230 | 346 | 2.2 |
| example 9′ | | | | | | | |
| Comparative | 0.7 | 9 | 35 | 0 | 230 | 323 | 2.1 |
| example 10′ | | | | | | | |

TABLE 9

| | | | | | | | | Amount of |
|---|---|---|---|---|---|---|---|---|
| | HEA | HOA-MS | PEG chain | PEG | AMPS | Primary particle | DLS | polymer |
| | (mL) | (μL) | length | (μL) | (mg) | diameter (nm) | (nm) | (wt %) |
| Example 22′ | 0.28 | 33 | 9 | 16 | 0 | 250 | 314 | 2.5 |
| Example 23′ | 0.23 | 27 | 9 | 13 | 0 | 250 | 304 | 2.7 |
| Example 24′ | 0.34 | 40 | 9 | 20 | 0 | 250 | 342 | 2.9 |
| Example 25′ | 0.17 | 20 | 9 | 10 | 0 | 250 | 284 | 2.0 |
| Example 26′ | 0.26 | 33 | 9 | 17 | 17 | 250 | 331 | 2.5 |

Considering the matter that the dispersion stability was very low due to too much amount of the deposited polymer in Comparative Example 6′ and too little amount of the deposited polymer in Comparative Example 7′, it was suggested that the magnetic particles were suitably prepared using appropriate amount of polymer raw materials as shown in Examples 1′ to 24′ according to the results of Tables 8 and 9; and the suitable molar ratio among the carboxyl group and the polyethylene glycol chain and the sulfo group were those shown in Examples 1′ to 26′.

[symbol]

Evaluation of Dispersion Stability in pH Buffer Liquid

[symbol]

### (Evaluation of Stability by Visual Observation)

Using each of the particles obtained from Examples 1′ and 13′ and Comparative Example 5′, the dispersion stability was evaluated. Water and PBS buffer liquid were used as a medium liquid. The concentration of the magnetic marker particles was adjusted to be 1 mg/ml. The dispersion was left for 10 minutes, thereafter the dispersion stability was evaluated based on the degree of its sedimentation. In the case where water was used, the degree of the dispersion stability were as follows:

(Example 1′) nearly equals to (Example 13′)>(Comparative Example 5′). However, the degree of the dispersion stability in the PBS buffer liquid was as follows: (Example 1′)>(Example 13′)>>>(Comparative Example 5′), wherein the differences had enlarged rather than the case of water. Accordingly, it was found that the dispersion stability of the magnetic particles increased in the case where the deposited polymer further contained the sulfo group or polyethylene glycol chain, rather than the case where the deposited polymer contained only the carboxyl group.

### (Evaluation of Dispersion Stability Based on Sedimentation Velocity)

Sedimentation rates in water and in phosphate buffered saline (PBS) were measured by using the particles obtained from Examples 1′, 5′, 8′, 12′, 22′ and 26′ as well as Comparative Examples 5′, 9′. As the measurement device, LUMiFuge 110 (manufactured by Nihon RUFUTO) was used. In the measurement condition, the speed of rotation was 500 rpm and the centrifugal force was 35×g in the measurement using PBS whereas the speed of rotation was 1000 rpm and the centrifugal force was 525×g in the measurement using water. The sample to be tested was introduced into the device and the change of the transmission factor (transmissivity) at each position in the cell was measured. Thereafter, the positional variation in the sample cell was obtained by the medium value of the transmission factor at the start of the measurement and at the end of the measurement. Based on the above, the value of sedimentation velocity V_{S} was calculated. Thereafter, the value V_{S} was divided by the centrifugal force so at to eliminate the influence of the centrifugal force, and thereby obtaining the sedimentation velocity of the present invention. That is, the sedimentation velocity V_{B} was calculated based on the above-mentioned Formula 1. The results are shown in Table 10.

TABLE 10

| | | Dispersion Medium: Water | | | | Dispersion Medium: PBS | | | | |
|---|---|---|---|---|---|---|---|---|---|---|
| | Primary | Centri- | Sedimentation | Sedimentation | Sedimen- | Centri- | Sedimentation | Sedimentation | Sedimen- | Ratio of |
| | particle | fugal | velocity | velocity V_{B} | tation | fugal | velocity | velocityV _{B} | tation | Sedimen- |
| | diameter | force A | Vs | in Formula 1 | velocity | force A | Vs | in Formula 1 | velocity | tation |
| | (nm) | (xg) | (μm/s) | (μm/sG) | V′ | (xg) | (μm/s) | (μm/sG) | V′ | velocity R |
| Example 1′ | 250 | 525 | 52.5 | 0.100 | 1.60E−06 | 35 | 69.4 | 1.98 | 3.17E−05 | 19.8 |
| Example 5′ | 140 | 525 | 45.5 | 0.087 | 4.44E−06 | 35 | 53.8 | 1.53 | 7.81E−05 | 17.6 |
| Example 8′ | 240 | 525 | 49.2 | 0.094 | 1.63E−06 | 35 | 65.3 | 1.87 | 3.25E−05 | 19.9 |
| Example 12′ | 250 | 525 | 66.3 | 0.126 | 2.02E−06 | 35 | 85.1 | 2.43 | 3.89E−05 | 19.3 |
| Example 22′ | 250 | 525 | 56.1 | 0.107 | 1.71E−06 | 35 | 30.7 | 0.930 | 1.49E−05 | 8.7 |
| Example 26′ | 250 | 525 | 59.2 | 0.113 | 1.80E−06 | 35 | 32.1 | 0.973 | 1.56E−05 | 8.6 |
| Comparative | 250 | 525 | 65.1 | 0.124 | 1.98E−06 | 35 | 170 | 4.86 | 7.78E−05 | 39.2 |
| example 5′ | | | | | | | | | | |
| Comparative | 250 | 5 | 252 | 50.4 | 8.06E−04 | 5 | 260 | 52.0 | 8.32E−04 | 1.0 |
| example 8′ | | | | | | | | | | |
| Comparative | 230 | 525 | 61.7 | 0.118 | 2.23E−06 | 35 | 71.1 | 2.03 | 3.84E−05 | 17.2 |
| example 9′ | | | | | | | | | | |

“OE-0X” denotes “OE × 10^{-x}” (For example, “OE-06” denotes “OE × 10⁻⁶”)

With reference to Table 10, it was found that each particle showed high dispersion stability in water. In the case of PBS, the value of V_{B} was generally low in Examples, so that the dispersion stability of Examples was high. On the other hand, in Comparative Examples 5′, 9′, the values of V_{B} in the case of PBS were relatively higher, so that the dispersion stability thereof was low. Thus, it can be understood that the magnetic marker particles of the present invention has high dispersion stabilities even in PBS.

[symbol]

Evaluation of Magnetic Collectivity

[symbol]

Using the particles obtained Example 1′ and Comparative Example 9′ as well as Dynabeads (MyOne Carboxylic acid (manufactured by Invitrogen Corporation), the magnetic collection rates were measured in water. As the measuring device, bio-spectrophotometer U-0080D (manufactured by Hitachi High-Technologies Corporation) was used. Specifically, a dispersion liquid of the magnetic particles (0.2 mg/mL) was introduced into a spectroscopic cell having 1 cm×1 cm square bottom, and the cell was placed in a spectrophotometer. After the particles were sufficiently dispersed by pipetting, a neodymium magnet NK037 (manufactured by Niroku Seisakusho) (outer size: 40 mm×20 mm×1 mm, surface magnetic flux density: 134 mT) was brought closer to the outside of the cell and measured the variation with time of the light absorbance at 550 nm. The magnetic field inside of the cell in this case was measured by the above-mentioned method. As a result, the value of the magnetic field was 0.36 T.

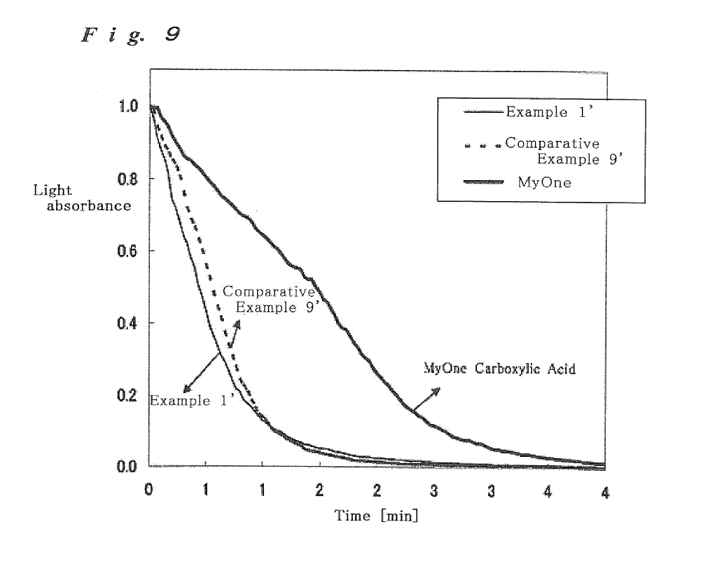

FIG. 9 shows the results of the measurement. As seen from FIG. 9, the order of the samples that shows rapid decrease in the light absorbance is (Example 1′)-(Comparative Example 9′)-(MyOne). Specifically, the relative light absorbance of the buffer solution decreased from its initial value “1” to about 0.15 in about 60 seconds after applying the magnetic field with respect to Example 1′ and Comparative Example 9′, wherein the rate of decrease in Example 1′ was faster or larger than that of Comparative Example 9′. That is, it was found that the magnetic marker particles of the present invention could be effectively magnetically collected in a shorter period of time in the dispersion liquid of the particles-containing buffer solution.

[symbol]

Evaluation of Re-Dispersibility

[symbol]

Evaluation tests were carried out in order to confirm the effects of “re-dispersibility (i.e. dispersibility or dispersion stability after the magnetic collection operation)”. Specifically, each particles of “Example 1′”, “raw material powder of Example 1′ (raw magnetic powder-1′)”, “particles obtained by subjecting the raw material powder of Example 1′ to the silane coupling agent treatment (Si treated powder-1′)”, “Comparative Example 9′”, “raw material powder of Example 5′ (raw magnetic powder-9′)”, and “particles obtained by subjecting the raw material powder of Example 5′ to the silane coupling agent treatment (Si treated powder-9′)” were dispersed in each solution of the phosphate buffered saline (PBS) (10 mg/ml). Each of the resultant buffer dispersions was subjected to the operation composed of “particles aggregation due to the magnetic collection” and “re-dispersion by using of microwave” at the following conditions, which operations was repeated ten times:
Magnetic collection operation: an operation of applying a magnetic field of 0.24 T to the whole buffer solution for 2 minutes (using a stand for separating magnetic beads “Magical Trapper” (manufactured by Toyobo Co., Ltd.), magnetic field measurement apparatus: “Handy Teslameter Elulu DTM6100” (manufactured by Mytech Corporation);
Ultrasonic irradiation operation (re-dispersion operation): an operation of applying ultrasonic energy to the “area of the aggregated magnetic marker particles” for 2 minutes using an ultrasonic cleaner (VS-150, frequency 50 kHz, output 150 W) (manufactured by As-One Corp.).

Before and after the above operations, the dispersion particle diameter (i.e., secondary particle diameter) was measured, thereby the degrees of the magnetic aggregation were compared. For the above measurement of the dispersion particle diameter, a laser diffraction/scattering particle size distribution analyzer LA-920 (manufactured by Horiba Ltd.) was used. It should be noted that the measurement of the above dispersion particle diameter was carried out using the DLS method, which was different from this laser diffraction/scattering particle size distribution analyzer LA-920 (manufactured by Horiba Ltd.). The reason for this is that the measurable range is in the range of a few nm to 5 μm in the DLS method, thus DLS method is considered not to be suitable for measuring the degree of the magnetic aggregation (since the measurement principles differ from each other, it often happens that different results are obtained depending on kinds of the measuring methods even if the same particles are used.).

The results of “evaluation of re-dispersibility” are shown in Table 11. In Table 11, respective particles of “Example 1”, “the raw magnetic powder-1′”, “Si treated powder-1′” (which are in the series of Example 1′), and “Comparative Example 9′”, “raw magnetic powder-9′” and “Si treated powder-9′)” (which are in the series of Comparative Example 9′) were compared with each other and evaluated. The standard deviation of the particle diameter expresses the width of the particle size distribution, wherein the larger standard deviation indicates the broader particle size distribution. It was found that “raw magnetic powder” and “Si treated-powder” had large average particle diameters and large particle size distributions before the magnetic collection, so that they had already formed broad aggregations and each of them tended to easily aggregate. On the other hand, Example 1′ and Comparative Example 9′ before the magnetic collection had narrow average particle diameters and narrow particle size distributions, so that the particles had fewer aggregations and tended to hardly aggregate. With regard to the distributions between before and after magnetization, Example 1′ and Comparative Example 9′ did not change, in contrast, the raw magnetic powder and the Si treated powder had enlarged.

The values of increase rate of the dispersion particle diameter upon magnetizing these particles were compared between the series of Example 1 and the series of Comparative Example 9′. As a result, in each of the combinations of Example 1′ and Comparative Example 9′; the raw magnetic powder-1′ and the raw magnetic powder-9′; and the Si treated powder-1′ and the Si treated powder-9′, the series of Example 1′ showed smaller value of increase rate. The reason for this seemed to be that the small coercive force was provided due to the shape of “sphere”, and thus the magnetic aggregates hardly formed.

The dispersion particle diameter had substantially no change in Example 1′ whereas that of the raw magnetic powder had enlarged by about 16%, and that of the Si treated powder had enlarged by about 7%. Namely, in the cases of the raw magnetic powder and the Si treated powder, the particles originally tended to easily aggregate and formed a broad aggregations, and furthermore the magnetic aggregations thereof had been promoted due to the magnetic field. The same was true for the series of Comparative Example 9′.

According to the above results, the magnetic marker particles of the present invention showed more desirable re-dispersibility than that of the conventional particles (Comparative Example 9′). It seems to be resulted from the matter that the particles had substantially spherical shape, thereby having smaller coercive force and thus hardly forming the magnetic aggregates. In addition, another matter that the polymer coating the surface of the magnetic marker particles had high steric hindrance seems to be another factor. That is, it is conceivable that, together with the matter that the force for forming the magnetic aggregates weakened, the force for suppressing the aggregation of the particles was larger than the force for forming the aggregation of the particles, thereby an effect to effectively suppress the aggregation was exerted.

TABLE 11

| | | Diameter of | Increase | |
|---|---|---|---|---|
| | | dispersed | rate of | |
| | | particles | diameter | Primary |
| | Magnetization | (avarage | of dispersed | particle |
| | (performed ten | diameter) | particles | diameter |
| Sample | times) | [μm] | (%) | [μm] |
| Example 1′ | Before | 0.79 ± 0.24 | 1.4 | 0.25 |
| | magnetization | | | |
| | After | 0.78 ± 0.24 | | |
| | magnetization | | | |
| Raw magnetic | Before | 1.82 ± 0.64 | 16.5 | 0.25 |
| powder -1′ | magnetization | | | |
| | After | 2.12 ± 0.69 | | |
| | magnetization | | | |
| Si-treated | Before | 1.26 ± 0.56 | 7.1 | 0.25 |
| powder -1′ | magnetization | | | |
| | After | 1.35 ± 0.57 | | |
| | magnetization | | | |
| Comparative | Before | 0.54 ± 0.23 | 3.7 | 0.23 |
| example 9′ | magnetization | | | |
| | After | 0.56 ± 0.23 | | |
| | magnetization | | | |
| Raw magnetic | Before | 1.41 ± 0.69 | 19.1 | 0.23 |
| powder -9′ | magnetization | | | |
| | After | 1.68 ± 0.76 | | |
| | magnetization | | | |
| Si-treated | Before | 1.16 ± 0.58 | 8.6 | 0.23 |
| powder -9′ | magnetization | | | |
| | After | 1.26 ± 0.60 | | |
| | magnetization | | | |

[symbol]

Immobilization Test of Biomaterial-Binding Material

[symbol]

Streptavidin was immobilized on the magnetic marker particles of Examples 1′ and 12′ and Comparative Example 9′. Specifically, in each of Examples 1′ and 10′ and Comparative Example 9′, the polymer coated magnetic particles obtained therefrom (each 2 mg) were dispersed in 1 mL of 10 mM phosphate buffer liquid (pH7.2) to obtain 1 ml of polymer coated magnetic particles liquid. Then, to the obtained particles liquid, 1 mL of solution in which 5 mg of DMT-MM (coupling agent) was dissolved in 1 ml of 10 mM phosphate buffer liquid (pH7.2) was added to form 2 mL of liquid, and then supersonic was applied thereto for 5 minutes, followed by being stirred at 1000 rpm for 25 minutes. Then, the magnetic separation was performed, and thereafter the supernatant liquid was removed and added 1 mL of 10 mM phosphate buffer liquid (pH7.2). Then, after pipetting, the resulting liquid was subjected to the supersonic washing for 1 minute, and the supernatant liquid was removed by the magnetic separation. These supersonic washing and magnetic separation were repeated once again. The resulting liquid was adjusted to have a volume of 1 mL by adding 10 mM phosphate buffer liquid (pH7.2) thereto, and thereby there was obtained a liquid wherein carboxyl group activated polymer coated magnetic particles were contained.

Then, 1 mg of streptavidin (manufactured by Wako Pure Chemical Industries, Ltd.) was dissolved in 0.5 ml of 10 mM phosphate buffer liquid (pH 7.2). To the resulting liquid, 0.5 ml of the carboxyl group activated polymer coated magnetic particles liquid was added and then supersonic was applied to the liquid for 1 hour. Then, the liquid was stirred by rotator overnight, thereby a reaction for binding the streptavidin to the carboxyl group was performed. After the completion of the reaction, the liquid was subjected to the magnetic separation, and the supernatant liquid was removed and then 1 mL of 10 mM phosphate buffer liquid (pH7.2) was added thereto. Then, after pipetting, the liquid was subjected to the supersonic washing for 1 minute, and the supernatant liquid was removed by the magnetic separation. To the resulting liquid, 1 ml of 0.2M Tris-HCl was added, and supersonic was applied thereto for 1 minute. Subsequently, the liquid was stirred by the rotator for 2 hours, thereby the unreacted activated carboxyl group was hydroxylated. After the reaction was completed, the liquid was subjected to the magnetic separation, and then the supernatant liquid was removed and 1 mL of 10 mM phosphate buffer liquid (pH7.2) was added. After pipetting, the liquid was subjected to the supersonic washing for 1 minute, and the supernatant liquid was removed by the magnetic separation. Such washing treatment was further twice repeated. The resulting liquid was adjusted to have a volume of 1 mL by adding 10 mM phosphate buffer liquid (pH7.2) thereto, thereby streptavidin-immobilized polymer coated magnetic particles liquid was obtained.

### (Evaluation Test of Specific Binding Ability)

In order to evaluate the specific binding ability between the streptavidin-immobilized polymer coated magnetic particles and biotin, the biotin-bound amount of the streptavidin-immobilized polymer coated magnetic particles was evaluated by using biotinylated HRP.

First, in each of Examples 1′ and 12′ and Comparative Example 9′, the polymer coated magnetic particles obtained therefrom were subjected to a streptavidin-immobilization treatment. The streptavidin-immobilized polymer coated magnetic particles were then dispersed in 0.05 mg/ml of PBS buffer liquid. 0.25 mL of the resulting dispersion liquid was introduced to the Eppendorf tube (1.5 mL). The supernatant was removed by the magnetic separation process, and 100 μl of biotinylated HRP (concentration: 100 ng/ml) was added to the liquid. The resulting liquid was stirred by the vortex mixer for 30 minutes, thereby the biotinylated HRP was immobilized to the streptavidin-immobilized polymer coated magnetic particles. The particles contained in the tube was washed with 400 μL of 10 mM PBS buffer liquid (pH7.2) and magnetically separated. This washing treatment was repeated four times in total. After removing PBS buffer solution (pH7.2), 200 μL of TMB (tetramethylbenzene) was added to the tube where the above particles were present, and left for 30 minutes, thereby developed the color of the particle liquid. The reaction was stopped by adding 200 μL of 1N sulfuric acid. The reaction-stopped liquid was diluted with 1N sulfuric acid to 5 fold, and 100 μL thereof was dispensed on a well plate. The degree of color development of the particles introduced from tubes was obtained by measuring the light absorbance (450 nm) thereof by the plate reader (infinite F200 (manufactured by TECAN)). The results are shown in Table 12.

TABLE 12

| | Light absorbance |
|---|---|
| | [—] |
| Example 1′ | 0.8 |
| Example 12′ | 0.8 |
| Comparative | 0.7 |
| example 9′ | |

According to the results shown in Table 12, the magnetic marker particles each having a spherical shape of Examples 1′ and 12′ were found to have higher biotin bing ding ability per a unit weight, rather than that of the magnetic particles of Comparative Example 9′. That is, it can be understood that the magnetic marker particles of the present invention are suitably available as a marker used in the biotechnological field or life-science field.

### INDUSTRIAL APPLICABILITY

The magnetic marker particle of the present invention exhibits a high dispersibility and dispersion stability in a pH buffer solution. Especially in a preferred embodiment, the magnetic marker particle of the present invention exhibits not only a practically satisfactory dispersion stability but also a practically satisfactory magnetic collectivity in a pH buffer solution, and therefore can be not only desirably used as a marker for detecting target biomaterials in the biotechnological field or the life-science field, but also can be used for various treatments such as a quantitative determination, a qualitative analysis, a separation and a purification of cells, proteins, nucleic acids and other biomaterials.

### CROSS REFERENCE TO RELATED PATENT APPLICATION

The present application claims the rights of priorities of Japan patent application No. 2010-127728 (filing date: Jun. 3, 2010, title of the invention: SPHERICAL MAGNETIC MARKER PARTICLE ANN METHOD FOR PRODUCING THE SAME) and Japan patent application No. 2010-127731 (filing date: Jun. 3, 2010, title of the invention: MAGNETIC MARKER PARTICLE WITH HIGH DISPERSION STABILITY AND MAGNETIC COLLECTIVITY), the whole contents of which are incorporated herein by reference.

### EXPLANATION OF REFERENCE NUMERALS

10 Cell for measurement
20 Magnet
30 Sensor for measuring magnetic field

What is claimed is:

1. A magnetic marker particle comprising a magnetic particle and a polymer deposited on the surface of the magnetic particle,
wherein the polymer comprises a combination of a carboxyl group and a polyethylene glycol chain or a combination of a carboxyl group and a sulfo group.

2. The magnetic marker particle according to claim 1, wherein a value of sedimentation velocity V_{B} represented by the following Formula 1 with regard to a buffer solution that contains the magnetic marker particle is in the range of 5.0×10⁻³ to 6.0:
V_{B}=V_{S}/A (Formula 1)
wherein
V_{B} [μm/(s·G)]: Sedimentation velocity of magnetic marker particle in buffer solution;
A[G]: Centrifugal force applied to buffer solution; and
V_{S} [μm/s]: Sedimentation velocity of magnetic marker particle in buffer solution upon applying centrifugal force A thereto.

3. The magnetic marker particle according to claim 1, wherein the magnetic marker particle has a spherical shape wherein a ratio of the largest radius to the smallest radius regarding a primary particle thereof is in the range of 1.0 to 1.3.

4. The magnetic marker particle according to claim 3, wherein, Coefficient of Variation (CV value) with regard to the spherical magnetic particles, which represents a distribution of their particle diameters, is not more than 18%.

5. The magnetic marker particle according to claim 2, wherein a sedimentation velocity ratio R represented by the following Formula 2 is in the range of 1.0 to 18, the ratio being obtained by dividing the value of sedimentation velocity V_{B} of the magnetic marker particle in a case of buffer solution by the value of sedimentation velocity V_{W} of the magnetic marker particle in a case of water:
R=V_{B}/V_{W} (Formula 2)
wherein
R[−]: Ratio of sedimentation velocity value of magnetic marker particle contained in buffer solution to sedimentation velocity value of magnetic marker particle contained in water;
V_{B} [μm/(s·G)]: Sedimentation velocity of magnetic marker particle contained in buffer solution; and
V_{W} [μm/(s·G)]: Sedimentation velocity of magnetic marker particle contained in water.

6. The magnetic marker particle according to claim 1, wherein a value of sedimentation velocity V′ represented by the following Formula 3 with regard to a buffer solution that contains the magnetic marker particle is in the range of 1.0×10⁻⁶ to 1.0×10⁻⁴:
V′=V_{S}/(A×D²) (Formula 3)
wherein
V′ [T/m·s·G]=[10¹²/m·s·G]: Sedimentation velocity of magnetic marker particle in buffer solution;
D [nm]: Diameter of magnetic marker particle as primary particle;
A[G]: Centrifugal force applied to buffer solution; and
V_{S} [μm/s]: Sedimentation velocity of magnetic marker particle in buffer solution upon applying centrifugal force A thereto.

7. The magnetic marker particle according to claim 1, wherein the polymer comprises the carboxyl group, the polyethylene glycol chain and the sulfo group.

8. The magnetic marker particle according to claim 1, wherein the amount of the polymer is in the range of 1 to 20% by weight based on the weight of the magnetic marker particle.

9. The magnetic marker particle according to claim 1, wherein the magnetic marker particle is a ferromagnetic particle.

10. The magnetic marker particle according to claim 1, wherein the magnetic particle comprises ferrite or magnetite.

11. The magnetic marker particle according to claim 1, wherein a biomaterial-binding material or biomaterial-binding functional group is immobilized on the magnetic particle and/or the polymer.

12. The magnetic marker particle according to claim 1, wherein the magnetic marker particle, as a primary particle, has a diameter of 20 nm to 600 nm.

13. The magnetic marker particle according to claim 3, wherein a saturation magnetization of the magnetic marker particle is in the range of 2 to 100 A·m²/kg (emu/g).

14. The magnetic marker particle according to claim 3, wherein a coercive force of the magnetic marker particle is in the range of 0.3 kA/m to 6.5 kA/m.

15. The magnetic marker particle according to claim 1, wherein, with respect to a buffer solution containing the magnetic marker particles (dispersion particle diameter of the magnetic marker particles: 200 nm to 700 nm, concentration of magnetic marker particles: 0.1 to 0.3 mg/mL), a time required for relative light absorbance of the buffer solution to become 0.1 to 0.2 (from an initial value being 1 before the following magnetic collection) upon magnetically collecting the magnetic marker particles in the buffer solution under the magnetic field of 0.36 T is within 60 seconds.

16. The magnetic marker particle according to claim 1, wherein an increase rate of a dispersion particle diameter of the magnetic marker particles contained in a buffer solution is within 5% with respect to the dispersion particle diameter of the magnetic particles contained in the before-treatment buffer solution, provided that such a treatment that the magnetic marker particles are dispersed in the buffer solution by an ultrasonic irradiation after being magnetically collected is repeated ten times.

17. A method for producing the magnetic marker particle as claimed in claim 7, comprising the step of depositing a polymer on the magnetic particle by the use of a polymer raw material,
wherein the polymer raw material comprises “compound with a polymerizable moiety and a carboxyl group therein”, “compound of a polyethylene glycol chain with at least two polymerizable moieties therein” and “compound with a polymerizable moiety and a sulfo group therein”.

18. The method according to claim 17, wherein the “compound with a polymerizable moiety and a carboxyl group therein” is an acrylic acid, and the “compound with a polymerizable moiety and a sulfo group therein” is a styrenesulfonic acid or a 2-acrylamido-2-methylpropanesulfonic acid.

19. The method according to claim 17, comprising immobilizing a biomaterial-binding material or biomaterial-binding functional group on the magnetic particle and/or the polymer.

20. The method for producing the magnetic marker particle as claimed in claim 1, wherein the magnetic particle serving as a core particle is prepared by a treatment comprising the steps of:
(i) mixing an iron-containing solution with an alkaline solution, thereby precipitating an iron element-containing hydroxide in the resulting mixture solution; and
(ii) subjecting the mixture solution to a heat treatment, thereby forming magnetic particle from the hydroxide.

21. The method according to claim 20, wherein, in the step (ii), the hydroxide is subjected to a solvothermal reaction in the mixture solution which comprises water and glycerin.

22. The method according to claim 20, wherein the mixture solution is irradiated with microwave in the heat treatment of the step (ii).
